(19) United States
(12) Patent Application Publication
(10) Pub. No.: US 2006/0025393 A1
(43) Pub. Date: Feb. 2, 2006

(54) Steroid derivatives

(76) Inventors: Shutsung Liao, Chicago, IL (US); Junichi Fukuchi, Matsumoto-shi (JP)

Correspondence Address: FISH & RICHARDSON PC, P.O. BOX 1022, MINNEAPOLIS, MN, 55440-1022, US

(21) Appl. No.: 10/962,199

(22) Filed: Oct. 8, 2004

Related U.S. Application Data
(63) Continuation-in-part of application No. 10/705,398, filed on Nov. 10, 2003.
Application No. 10/705,398 is a continuation of application No. 09/560,236, filed on Apr. 28, 2000, now Pat. No. 6,645,955.
(60) Provisional application No. 60/131,728, filed on Apr. 30, 1999.
(60) Provisional application No. 60/191,864, filed on Mar. 24, 2000.

(51) Int. Cl. A61K 31/56

(52) U.S. Cl. 514/182

(57) ABSTRACT

This invention relates to methods for treating cancers.

### CROSS-REFERENCE TO RELATED APPLICATIONS

This application is a continuation-in-part of U.S. application Ser. No. 10/705,398, filed on Nov. 10, 2003, which is a continuation of U.S. application Ser. No. 09/560,236, filed on Apr. 28, 2000, which claims the benefit of prior U.S. Provisional Application 60/131,728, filed on Apr. 30, 1999; and U.S. Provisional Application 60/191,864, filed on Mar. 24, 2000. The contents of each of these prior applications are incorporated herein by reference in their entireties.

### STATEMENT AS TO FEDERALLY SPONSORED RESEARCH

This invention was made with Government support under NIH CA 58073 and NIH AT00850 from National Institute of Health. The Government may therefore have certain rights in this invention.

### TECHNICAL FIELD

This invention relates to methods for treating cancers.

### BACKGROUND

Prostate cancer is the most commonly diagnosed malignancy and the second leading cause of cancer death among American men. In general, prostate tumors are initially dependent on androgen for growth, even after metastasis, and therefore can be treated effectively by androgen deprivation. Prostate tumors can reappear, typically after 1 to 3 years of endocrine therapy, as androgen-independent tumors. Androgen deprivation or antiandrogen therapies are generally ineffective against androgen-independent tumors.

The normal prostate produces and secretes a relatively significant amount of cholesterol in prostatic fluid. In benign prostatic hypertrophy and prostatic adenocarcinorma, the levels of tissue and secreted cholesterol are two to ten fold higher than in healthy prostate. It has also been reported that sterol response element binding proteins (SREBPs), transcriptional regulators that control the metabolic pathway of lipogenesis and cholesterol, are activated in androgen-independent tumors.

Liver X receptors (LXRs), e.g., LXRα and LXRβ, are nuclear receptors, which are believed to function as central transcriptional regulators for lipid homeostasis. LXRs are believed to function as heterodimers with retinoid X receptors (RXRs), and these dimers can be activated by ligands for either receptor. LXRα is expressed at relatively high levels in liver, intestine, adipose tissue and macrophages, whereas LXRβ is expressed ubiquitously and has been dubbed the ubiquitous receptor (UR). LXR response elements in LXR-target genes are direct repeats of the consensus AGGTCA separated by four nucleotides. Both LXRs in macrophages are believed to control the cholesterol efflux pathway through the regulation of target genes including ATP-binding cassette A1 (ABCA1) and apolipoprotein E.

### SUMMARY

In one aspect, this invention relates to steroid derivatives of formula (I):

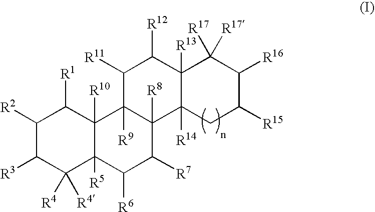

R³ is hydrogen, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid.

Each of R¹, R², R⁴, R^{4′}, R⁶, R⁷, R¹¹, R¹², R¹⁵, R¹⁶, and R^{17′}, independently, is hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid.

Each of R⁵, R⁸, R⁹, R¹⁰, R¹³, and R¹⁴, independently, is hydrogen, alkyl, haloalkyl, hydroxyalkyl, alkoxy, hydroxy, or amino.

R¹⁷ is —X—Y-Z. X is a bond, or alkyl or alkenyl, optionally inserted with —NH—, —N(alkyl)-, —O—, or —S—, and further optionally forming a cyclic moiety with R¹⁶ and the 2 ring carbon atoms to which R¹⁶ and R¹⁷ are bonded. Y is —CO—, —SO—, —SO₂—, —O—SO₂—, —SO₂—O—, —O—SO₃—, —SO₃—O—, —CO—O—, —O—CO—, —CO—NH—, —CO—N(alkyl)-, —NH—CO—, —N(alkyl)-CO—, or a bond. Z is alkyl, alkenyl, alkynyl, cycloalkyl, heterocycloalkyl, cycloalkenyl, heterocycloalkenyl, aryl, heteroaryl, aralkyl, or heteroaralkyl, and is optionally substituted with hydroxy, alkoxy, amino, halo, sulfonic acid, —O-sulfonic acid, carboxyl, oxo, alkyloxycarbonyl, alkylcarbonyloxy, alkylaminocarbonyl, alkylcarbonylamino, alkylcarbonyl, alkylsulfinyl, alkylsulfonyl, or alkylthio; or is —CH(A)-B. A being a side chain of an amino acid, and B is hydrogen, —NR^{a}R^{b}, or —COOR^{c} wherein each of R^{a}, R^{b}, and R^{c}, independently, is hydrogen or alkyl.

n is 0, 1, or 2.

In some embodiments, when Z is substituted with carboxyl or alkyloxycarbonyl, Y is a bond and either X or Z contains at least one double bond, and that when Y is a bond, either X is —NH-alkyl-, —NH-alkenyl-, —N(alkyl)-alkyl-, —N(alkyl)-alkenyl-, —O-alkyl-, —O-alkenyl-, —S-alkyl-, or —S-alkenyl-; or Z is substituted with halo, sulfonic acid, —O-sulfonic acid, alkylsulfinyl, or alkylsulfonyl, or is alkenyl.

In another aspect, this invention relates to steroid derivatives having the formula (I) as depicted above.

Each of R¹, R², R³, R⁴, R⁴′, R⁶, R⁷, R¹¹, R¹², R¹⁵, R¹⁶, and R¹⁷′, independently, is hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid.

Each of R⁵, R⁸, R⁹, R¹⁰, R¹³, and R¹⁴, independently, is hydrogen, alkyl, haloalkyl, hydroxyalkyl, alkoxy, hydroxy, or amino.

R¹⁷ is —X—Y-Z. X is a bond, or alkyl or alkenyl, optionally inserted with —NH—, —N(alkyl)-, —O—, or —S—, and further optionally forming a cyclic moiety with R¹⁶ and the 2 ring carbon atoms to which R¹⁶ and R¹⁷ are bonded. Y is —CO—, —SO—, —SO₂—, —O—SO₂—, —SO₂—O—, —O—SO₃—, —SO₃—O—, —CO—O—, —O—CO—, —CO—NH—, —CO—N(alkyl)-, —NH—CO—, —N(alkyl)-CO—, or a bond. Z is alkyl, alkenyl, alkynyl, cycloalkyl, heterocycloalkyl, cycloalkenyl, heterocycloalkenyl, aryl, heteroaryl, aralkyl, or heteroaralkyl, and is optionally substituted with hydroxy, alkoxy, amino, halo, sulfonic acid, —O-sulfonic acid, carboxyl, oxo, alkyloxycarbonyl, alkylcarbonyloxy, alkylaminocarbonyl, alkylcarbonylamino, alkylcarbonyl, alkylsulfinyl, alkylsulfonyl, or alkylthio; or is —CH(A)-B. A is an amino acid side chain containing an aromatic moiety, and B is hydrogen, —NR^{a}R^{b}, or —COOR^{c} wherein each of R^{a}, R^{b}, and R^{c}, independently, is hydrogen or alkyl.

n is 0, 1, or 2.

In some embodiments, when Z is substituted with carboxyl or alkyloxycarbonyl, Y is a bond and either X or Z contains at least one double bond, and that when Y is a bond, either X is —NH-alkyl-, —NH-alkenyl-, —N(alkyl)-alkyl-, —N(alkyl)-alkenyl-, —O-alkyl-, —O-alkenyl-, —S-alkyl-, or —S-alkenyl-; or Z is substituted with halo, sulfonic acid, —O-sulfonic acid, alkylsulfinyl, or alkylsulfonyl, or is alkenyl.

In a further aspect, this invention relates to steroid derivatives of formula (I), supra. Each of R¹, R², R³, R⁴, R^{4′}, R⁶, R⁷, R¹¹, R¹², R¹⁵, R¹⁶, and R^{17′}, independently, is hydrogen, hydroxy, amino, carboxyl, oxo, halo, sulfonic acid, —O-sulfonic acid, or alkyl optionally inserted with —NH—, —N(alkyl)-, —O—, —S—, —SO—, —SO₂—, —O—SO₂—, —SO₂—O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid.

Each of R⁵, R⁸, R⁹, R¹⁰, R¹³, and R¹⁴, independently, is hydrogen, alkyl, haloalkyl, hydroxyalkyl, alkoxy, hydroxy, or amino.

R¹⁷ is —X—Y-Z. X is a bond, or alkyl or alkenyl, optionally inserted with —NH—, —N(alkyl)-, —O—, or —S—, and further optionally forming a cyclic moiety with R¹⁶ and the 2 ring carbon atoms to which R¹⁶ and R¹⁷ are bonded. Y is —CO—, —SO—, —SO₂—, —O—SO₂—, —SO₂—O—, —O—SO₃—, —SO₃—O—, —CO—O—, —O—CO—, —CO—NH—, —CO—N(alkyl)-, —NH—CO—, —N(alkyl)-CO—, or a bond. Z is alkyl, alkenyl, alkynyl, cycloalkyl, heterocycloalkyl, cycloalkenyl, heterocycloalkenyl, aryl, heteroaryl, aralkyl, or heteroaralkyl, and is optionally substituted with hydroxy, alkoxy, amino, halo, sulfonic acid, —O-sulfonic acid, carboxyl, oxo, alkyloxycarbonyl, alkylcarbonyloxy, alkylaminocarbonyl, alkylcarbonylamino, alkylcarbonyl, alkylsulfinyl, alkylsulfonyl, or alkylthio; or is —CH(A)-B. A is a side chain of an amino acid, and B is hydrogen, —NR^{a}R^{b}, or —COOR^{c} wherein each of R^{a}, R^{b}, and R^{c}, independently, is hydrogen or alkyl.

n is 0, 1, or 2.

In some embodiments, when Z is substituted with carboxyl or alkyloxycarbonyl, Y is a bond and either X or Z contains at least one double bond, and that when Y is a bond, either X is —NH-alkyl-, —NH-alkenyl-, —N(alkyl)-alkyl-, —N(alkyl)-alkenyl-, —O—alkyl-, —O-alkenyl-, —S-alkyl-, or —S-alkenyl-; or Z is substituted with halo, sulfonic acid, —O-sulfonic acid, alkylsulfinyl, or alkylsulfonyl, or is alkenyl; and that at least one of R³ and R⁴, R⁴ and R⁵, R⁵ and R₆, R⁷ and R⁸, R¹² and R¹³, and R¹⁵ and R¹⁶, independently, is deleted to form a double bond.

One subset of the just-described steroid derivatives encompasses compounds which are featured by the presence of at least one double bond in the rings, which are formed by deleting one or more of the following pairs of substituents: R³ and R⁴, R⁴ and R⁵, R¹² and R¹³, and R¹⁵ and R¹⁶. Another subset encompasses compounds which are featured by that Z is alkenyl, alkynyl, cycloalkyl, heterocycloalkyl, cycloalkenyl, heterocycloalkenyl, aryl, heteroaryl, aralkyl, or heteroaralkyl, and optionally substituted with hydroxy, alkoxy, amino, or halo; or is —CH(A)-B. A and B are as described above.

Note that X and Z can optionally join together to form a cyclic moiety. For example, if both X and Z are alkyl, and Y is —C(═O)—O—, a lactone results from joining X and Z.

A salt of the steroid derivative of this invention is also within the scope of this invention and can be formed, for example, between the steroid of this invention having a carboxylate and a cationic counterion such as an alkali metal cation, e.g., a sodium ion or a potassium ion; or an ammonium cation that can be substituted with organic groups, e.g., a tetramethylammonium ion or a diisopropyl-ethylammonium ion. A salt of this invention can also form between the steroid derivative of this invention having a protonated amino group and an anionic counterion, e.g., a sulfate ion, a nitrate ion, a phosphate ion, or an acetate ion.

Set forth below are some examples of steroid derivatives of formula (I):

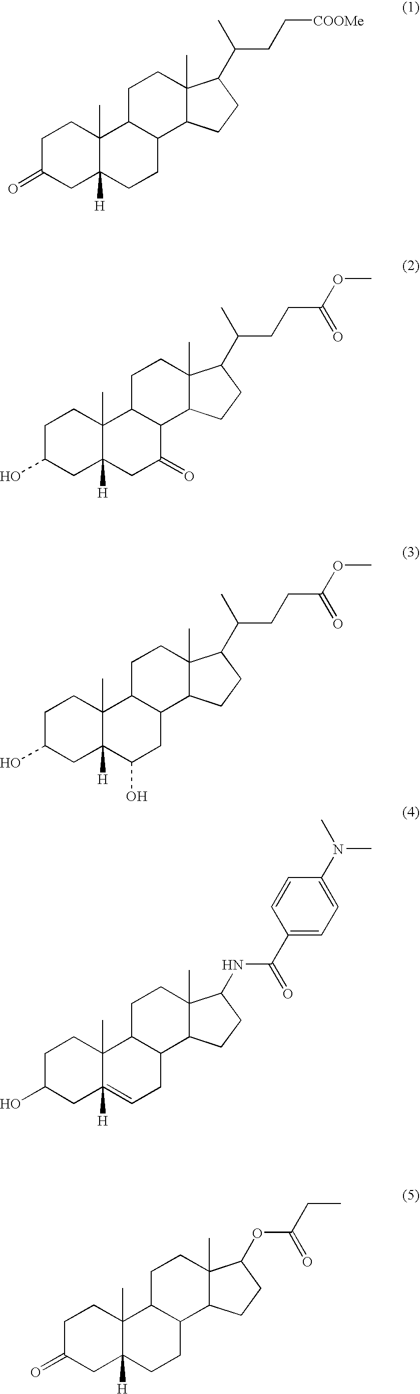

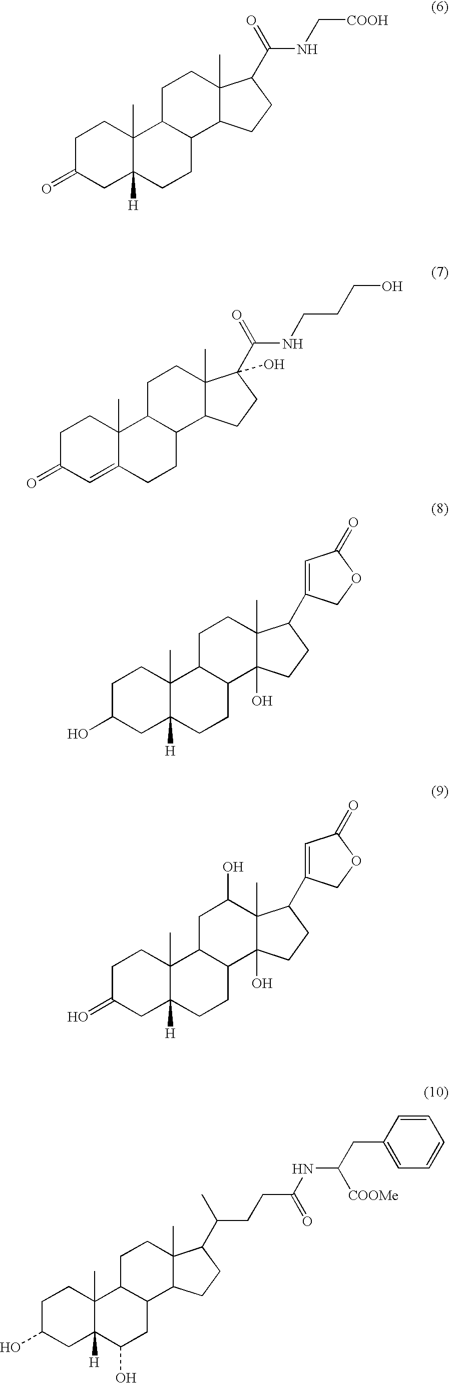

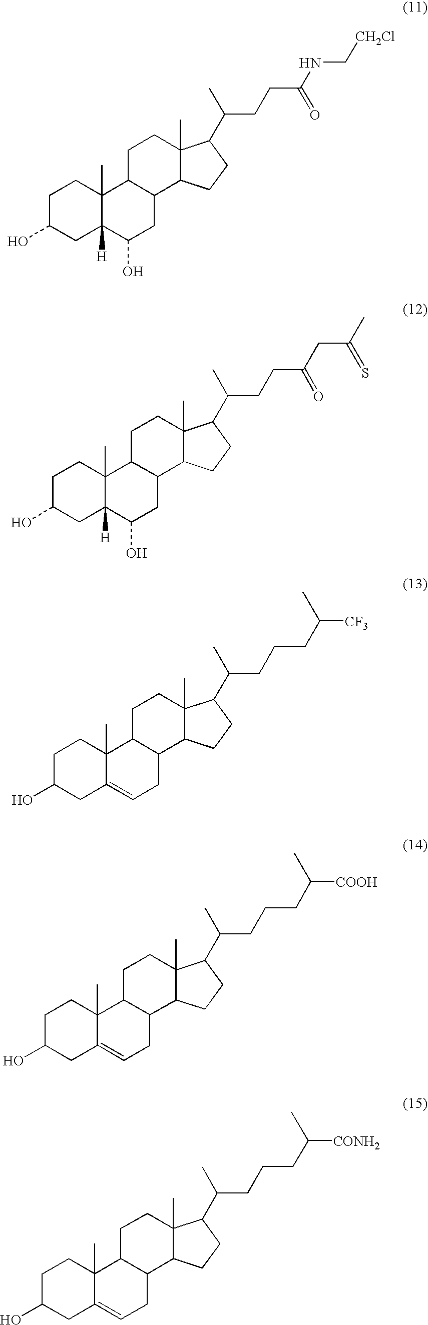

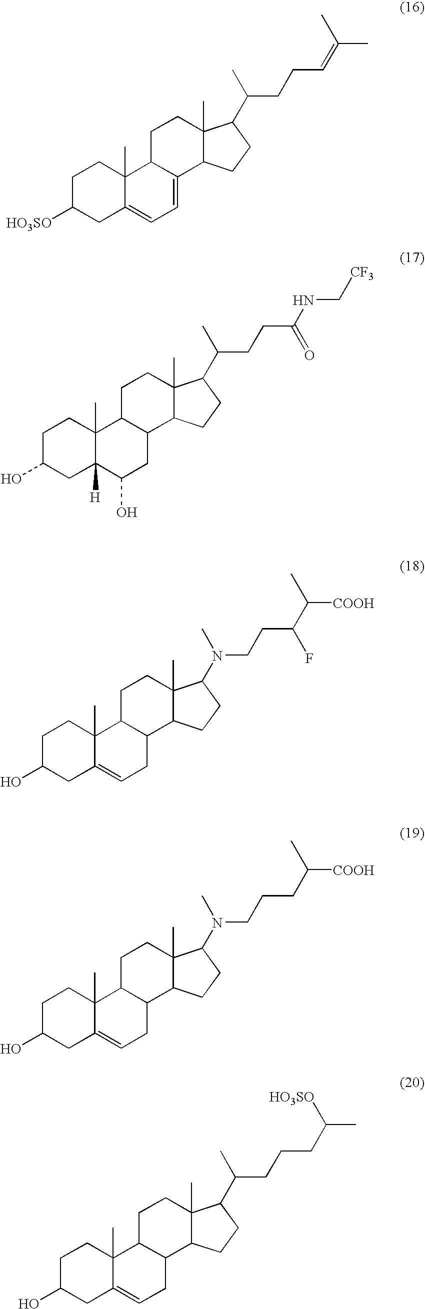

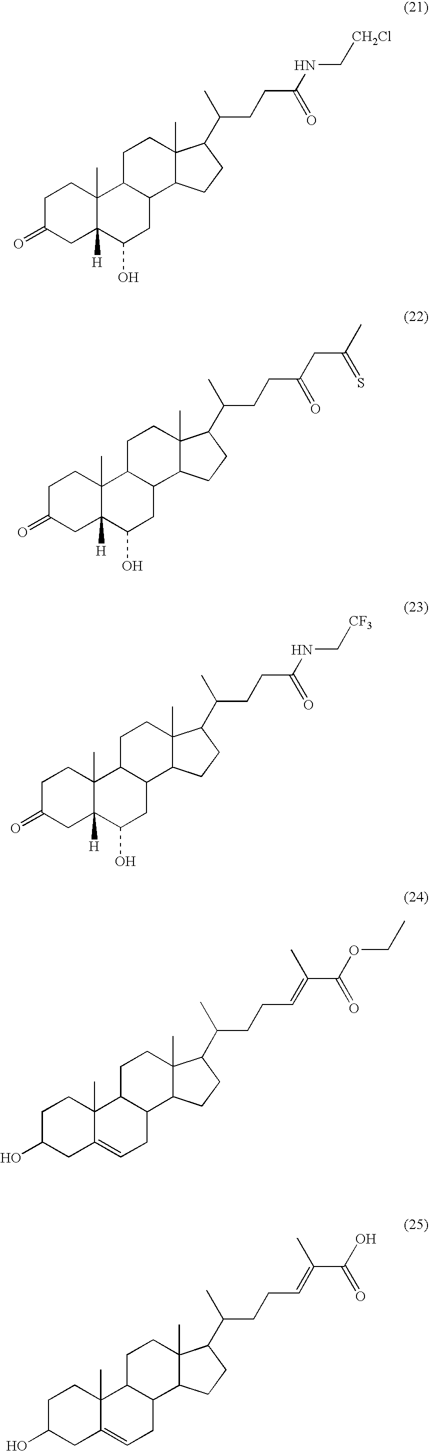

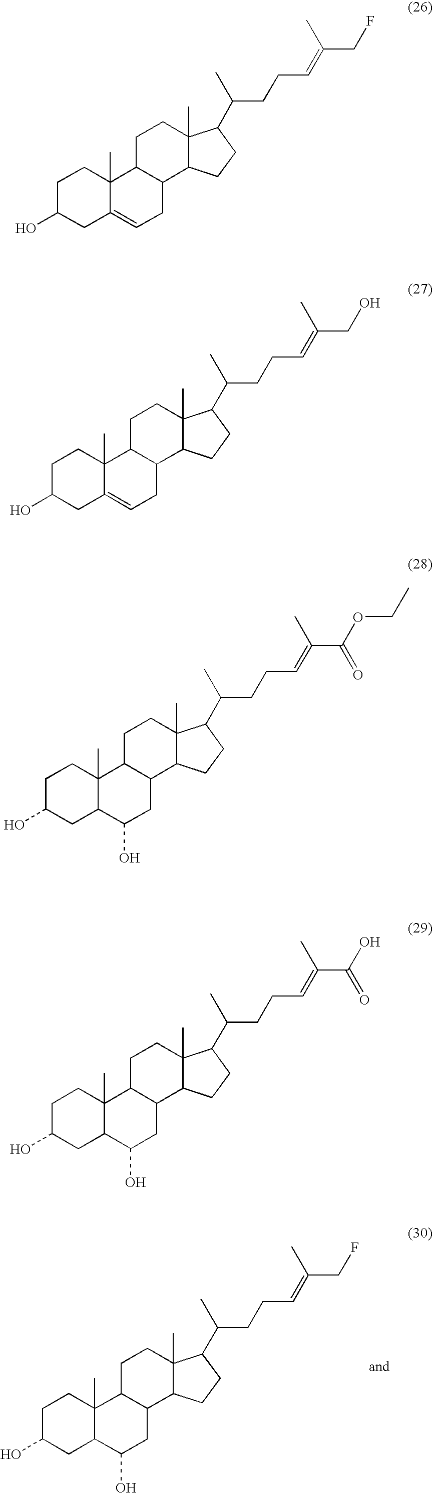

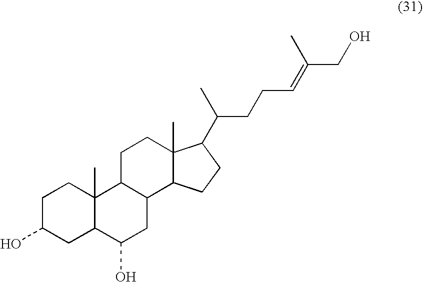

Yet another aspect of this invention relates to a pharmaceutical composition for treating a UR- or LXRa-mediated disorder which contains a pharmaceutically acceptable carrier and an effective amount of one or more of the steroid derivatives described above. The use of such a steroid derivative or a salt thereof for the manufacture of a medicament for treating the above-mentioned disorders is also within the scope of this invention.

A still further aspect of this invention relates to a pharmacological composition for treating cancer, including solid tumors and leukemia, and immune dysfunction. The pharmacological composition contains a pharmaceutically acceptable carrier and an effective amount of one or more of a steroid derivative of formula (I), supra. Each of R¹, R², R³, R⁴, R^{4′}, R⁶, R⁷, R¹¹, R¹², R¹⁵, R¹⁶, and R^{17′}, independently, is hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —O—SO₃—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid. Each of R⁵, R⁸, R⁹, R¹⁰, R¹³, and R¹⁴, independently, is hydrogen, alkyl, haloalkyl, hydroxyalkyl, alkoxy, hydroxy, or amino. R¹⁷ is —X—Y-Z, in which X is a bond, or alkyl or alkenyl, optionally inserted with —NH—, —N(alkyl)-, —O—, or —S—, and further optionally forming a cyclic moiety with R¹⁶ and the 2 ring carbon atoms to which R¹⁶ and R¹⁷ are bonded; Y is —CO—, —SO—, —SO₂—, —O—SO₂—, —SO₂—O—, —O—SO₃—, —SO₃—O—, —CO—O—, —O—CO—, —CO—NH—, —CO—N(alkyl)-, —NH—CO—, —N(alkyl)-CO—, or a bond; and Z is alkyl, alkenyl, alkynyl, cycloalkyl, heterocycloalkyl, cycloalkenyl, heterocycloalkenyl, aryl, heteroaryl, aralkyl, or heteroaralkyl, and is optionally substituted with hydroxy, alkoxy, amino, halo, sulfonic acid, —O-sulfonic acid, carboxyl, alkyloxycarbonyl, alkylcarbonyloxy, alkylaminocarbonyl, alkylcarbonylamino, alkylcarbonyl, alkylsulfinyl, alkylsulfonyl, or alkylthio; or is —CH(A)-B with A being a side chain of an amino acid, and B being hydrogen, —NR^{a}R^{b}, or —COOR^{c} wherein each of R^{a}, R^{b}, and R^{c}, independently, is hydrogen or alkyl; and n is 0, 1, or 2. When Z is substituted with carboxyl, Y is a bond and either X or Z contains at least one double bond, and when Y is a bond, either X is —NH-alkyl-, —NH-alkenyl-, —N(alkyl)-alkyl-, —N(alkyl)-alkenyl-, —O-alkyl-, —O-alkenyl-, —S-alkyl-, or —S-alkenyl-; or Z is substituted with halo, sulfonic acid, —O-sulfonic acid, alkylsulfinyl, or alkylsulfonyl, or is alkenyl. The use of a just-described steroid derivative or a salt thereof for the manufacture of a medicament for treating the above-mentioned disorders is also within the scope of this invention.

Still another aspect of the present invention relates to a method of treating a UR- or LXRa-mediated disorder by administering to a patient in need thereof an effective amount of one of the pharmaceutical compositions described above. Some examples of UR- or LXRa-mediated disorders are: liver cirrhosis, gallstone disease, hyperlipoproteinemias, Alzheimer's disease, anemia, chronic inflammatory diseases (e.g., rheumatoid arthritis), metabolic disorders (e.g., diabetes), and cancers which are associated with UR expression, e.g., breast cancer, colon cancer, prostate cancer, and leukemia. Patients with other disorders such as atherosclerosis and liver cholestasis can also be treated with one of the pharmaceutical compositions described above.

In a further aspect, this invention also relates generally to inhibiting the proliferation of cancer cells with compounds of formula (I). In some embodiments, the methods can include in vitro methods, e.g., contacting a cell culture (e.g., representing one or more cancer cell lines) or a cancerous tissue (e.g., having one or more types of tumors) with a compound of formula (I). In other embodiments, the methods can include in vivo methods, e.g., administering a compound of formula (I) to a subject (e.g., a subject in need thereof, e.g., a mammal, e.g., a human). The cancer can be a cancer, which is associated with UR expression, e.g., breast cancer, colon cancer, prostate cancer, and leukemia.

Embodiments can include one or more of the following features.

The cancer can be a sex hormone-dependent cancer.

The sex hormone-dependent cancer can be prostate cancer. In some embodiments, the prostate cancer can be an androgen-dependent prostate cancer. In some embodiments, the prostate cancer can be resistant to androgen deprivation and/or antiandrogen therapies, (e.g., an androgen-independent prostate cancer, e.g., a hormone-refractory prostate cancer).

The subject can have at least one prostate cancer tumor that is resistant to androgen deprivation and/or antiandrogen therapies, e.g., an androgen-independent prostate cancer tumor. In some embodiments, the subject can further be substantially free of androgen-dependent prostate cancer tumors.

The sex hormone-dependent cancer can be breast cancer.

The Liver X receptor agonist can be orally administered.

The Liver X receptor can be LXRα or LXRβ.

n can be 0.

R³ can be amino, carboxyl, halo, sulfonic acid, —O-sulfonic acid, or alkyl; R⁶ is hydroxy, amino, carboxyl, halo, sulfonic acid, —O-sulfonic acid, or alkyl; and each of R³ and R⁶, independently, can be in the α-configuration.

R³ can be hydroxy, amino, carboxyl, halo, sulfonic acid, —O-sulfonic acid, or alkyl, and is in the α-configuration.

R⁵ can be hydrogen and can be in the β-configuration.

R³ can be oxo; each of R¹, R², R⁴, R^{4′},R⁶, R⁷, R¹¹, R¹², R¹⁵, R¹⁶, and R^{17,}, independently, can be hydrogen, hydroxy, oxo, halo, sulfonic acid, —O-sulfonic acid, or alkyl.

Each of R³ and R⁶, independently, can be hydroxy, amino, carboxyl, halo, sulfonic acid, —O-sulfonic acid, or alkyl, and is in the α-configuration.

Each of R¹, R², R⁴, R^{4′}, R⁶, R⁷, R¹¹, R¹², R¹⁵, R¹⁶, and R¹⁷′, independently, can be hydrogen, hydroxy, or oxo; and each of R⁵, R⁸, R⁹, R¹⁰, R¹³, and R ¹⁴, independently, can be hydrogen or hydroxy; or a salt thereof.

X can be a bond or alkyl.

Y can be —C(═O)—NH— or —NH—C(═O)—; and Z can be —CH(A)-B with A being a side chain of Tyr or Phe, and B being —NR^{a}R^{b} or —COOR^{c}.

Y can be —CO—, —O—SO₂—, —SO₂—O—, —O—SO₃—, —SO₃—O—, —CO—NH—, —NH—CO—, or a bond.

Z can be alkyl, alkenyl, aryl, heteroaryl, aralkyl, or heteroaralkyl, and is optionally substituted with hydroxy, alkoxy, halo, sulfonic acid, carboxyl, —O-sulfonic acid, alkylsulfinyl, or alkylsulfonyl; or can be —CH(A)-B.

Z can be alkyl or aryl, each of which being optionally substituted with hydroxy; or is —CH(A)-B with A being an amino acid side chain having an aromatic moiety, and B being —NR^{a}R^{b}, or —COOR^{c}.

R¹⁷ can contain a straight chain having 6-20 chain atoms.

R¹⁷ can contain a straight chain having 8-16 chain atoms.

X can be —CH(CH₃)—CH₂—, Y can be a bond, and Z can be —CH₂—CH═C(R′)(CH₃) with R′ being hydroxy, alkoxy, amino, halo, sulfonic acid, —O-sulfonic acid, carboxyl, oxo, alkyloxycarbonyl, alkylcarbonyloxy, alkylaminocarbonyl, alkylcarbonylamino, alkylcarbonyl, alkylsulfinyl, alkylsulfonyl, or alkylthio.

At least one of R³ and R⁴, R⁴ and R⁵, R¹² and R¹³, and R¹⁵ and R¹⁶, independently, can be deleted to form a double bond.

In one aspect, this invention relates to a method for treating cancer (e.g., a cancer, which is associated with UR expression, e.g., breast cancer, colon cancer, prostate cancer, and leukemia), the method includes administering to a subject (e.g., a subject in need thereof) an effective amount of a Liver X Receptor agonist having formula (II):

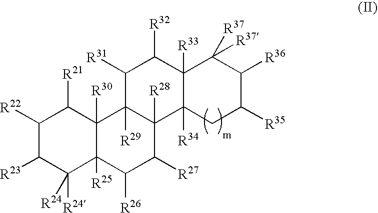

in which:

Each of R²¹, R²², R^{24′}, R³¹, and R^{37′}, independently, is hydrogen, halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, C(O)NR^{a}R^{b}; or —NR^{d}C(O)R^{c}.

Each of R²³, R²⁴, R²⁶, R²⁷, R³², R³⁵, R³⁶, independently, is hydrogen, halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, —C(O)NR^{a}R^{b}; or —NR^{d}C(O)R^{c}; or R²³ together with R²⁴ is a bond, R²⁴ together with R²⁵ is a bond, R²⁵ together with R²⁶ is a bond, R²⁷ together with R28 is a bond, R³² together with R³³ is a bond, or R³⁵ together with R³⁶ is a bond.

Each of R²⁵, R²⁸, and R³³, independently, is hydrogen or C₁-C₁₂ alkyl; or R²⁵ together with R²⁴ is a bond, R²⁵ together with R²⁶ is a bond, R²⁸ together with R²⁷ is a bond, or R³³ together with R³² is a bond.

Each of R²⁹, R³⁰, and R³⁴, independently, is hydrogen or C₁-C₁₂ alkyl.

R³⁷ is C₁-C₂₀ alkyl substituted with hydroxy, oxo, NR^{a}R^{b}, C₁-C₁₂ alkoxy, —C(O)R^{c}, —OC(O)R^{c}, or —NR^{d}C(O)R^{c}; or —C(O)NR^{a}R^{b}.

Each of R^{a}, R^{b}, and R^{d}, at each occurrence is, independently, hydrogen or C₁-C₁₀ alkyl.

R^{c}, at each occurrence is, independently, C₁-C₁₂ alkyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₆-C₁₆ aryl; or heteroaryl including 5-16 atoms; and

m is 0, 1, or 2; provided that at least one of R²³ and R²⁴, R²⁴ and R²⁵, R²⁵ and R²⁶, R²⁷ and R²⁸, R³² and R³³ or R³⁵ and R³⁶, together is a bond;

or a salt (e.g., a pharmaceutically acceptable salt) thereof.

In another aspect, this invention also relates generally to inhibiting the proliferation of cancer cells with compounds of formula (II). In some embodiments, the methods can include in vitro methods, e.g., contacting a cell culture (e.g., representing one or more cancer cell lines) or a cancerous tissue (e.g., having one or more types of tumors) with a compound of formula (II). In other embodiments, the methods can include in vivo methods, e.g., administering a compound of formula (II) to a subject (e.g., a subject in need thereof, e.g., a mammal, e.g., a human). The cancer can be a cancer, which is associated with UR expression, e.g., breast cancer, colon cancer, prostate cancer, and leukemia.

Embodiments can include one or more of the following features.

The cancer can be a sex hormone-dependent cancer.

The sex hormone-dependent cancer can be prostate cancer. In some embodiments, the prostate cancer can be an androgen-dependent prostate cancer. In some embodiments, the prostate cancer can be resistant to androgen deprivation and/or antiandrogen therapies, (e.g., an androgen-independent prostate cancer, e.g., a hormone-refractory prostate cancer).

The subject can have at least one prostate cancer tumor that is resistant to androgen deprivation and/or antiandrogen therapies, e.g., an androgen-independent prostate cancer tumor. In some embodiments, the subject can further be substantially free of androgen-dependent prostate cancer tumors.

The sex hormone-dependent cancer can be breast cancer.

The Liver X receptor agonist can be orally administered.

The Liver X receptor can be LXRα or LXRβ.

R²⁵ together with R²⁶ can be a bond.

m can be 0.

R²³ can be halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, —C(O)NR^{a}R^{b}; or —NR^{d}C(O)R^{c}.

R²³ can be hydroxy, oxo, —O-sulfonic acid, NR^{a}R^{b}, C₁-C₁₂ alkoxy, C₁-C₁₂ haloalkoxy, C₆-C₁₆ aryloxy, —OC(O)R^{c}, or —NR^{d}C(O)R^{c}.

R²³ can be hydroxy, oxo, —O-sulfonic acid, C₁-C12 alkoxy, C₁-C₁₂ haloalkoxy, C₆-C₁₆ aryloxy, or —OC(O)R^{c}.

R²³ can be hydroxy.

R²³ can be in the β-configuration.

Each of R²¹, R²², R²⁴, R^{24′}, R²⁷, R³¹ ,R³², R³⁵, R³⁶, and R^{37′}, independently, can be hydrogen, hydroxy, or oxo, and each of R²⁸, R²⁹, R³⁰, R³³, and R³⁴ can be hydrogen or C₁-C₆ alkyl.

Each of R²¹, R²², R²⁴, R^{24′}, R²⁷, R²⁸, R²⁹, R³¹, R³², R³⁴, R³⁵, R³⁶, and R^{37′} is hydrogen and each of R³⁰ and R³³ can be C₁-C₆ alkyl.

R²³ can be hydroxyl, each of R²¹, R²², R²⁴, R^{24′}, R²⁷, R²⁸, R²⁹, R³¹, R³², R³⁴, R³⁵, R³⁶, and R^{37′} can be hydrogen, and each of R³⁰ and R³³ can be C₁-C₆ alkyl.

Each of R³⁰ and R³³ can be CH₃.

R²³ can be in the β-configuration.

R³⁷ can be C₁-C₂₀ alkyl substituted with hydroxy (e.g., C₆-C₂₀ alkyl substituted with hydroxy, C₈-C₁₆ alkyl substituted with hydroxy).

R³⁷ can be —CH(CH₃)CH(OH)CH₂CH₂CH(CH₃)₂ or —CH(CH₃)CH₂CH₂CH(OH)CH(CH₃)₂.

The Liver X receptor agonist can be 22(R)-hydroxycholesterol or 24(S)-hydroxycholesterol.

The compound of formula (I) can be administered with a pharmaceutically acceptable carrier or adjuvant.

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In another aspect, this invention relates to a packaged product. The packaged product includes a container, a compound of formula (II) in the container, and a legend (e.g., a label or an insert) associated with the container and indicating administration of the compound for treatment of any of the cancers described herein.

In another aspect, the invention relates to a compound (including a pharmaceutically acceptable salt thereof) of formula (II), or a composition comprising a compound (including a pharmaceutically acceptable salt thereof) of formula (II). In some embodiments, the composition can further include a pharmaceutically acceptable adjuvant, carrier or diluent and/or an additional therapeutic agent.

The use of a compound of formula (II) or a salt thereof for the manufacture of a medicament for treating cancer is also within the scope of this invention.

The term “mammal” includes organisms, which include mice, rats, cows, sheep, pigs, rabbits, goats, and horses, monkeys, dogs, cats, and humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 to about 100 mg/Kg, from about 1 to about 100 mg/Kg). In certain embodiments, the dosage can be about 10 mg/Kg daily. Effective doses will also vary depending on route of administration, as well as the possibility of co-usage with other agents. The effective amount to be administered to a patient is typically based on body surface area, patient weight, and patient condition.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be substituted. Examples of alkyl groups include without limitation methyl, ethyl, propyl, isopropyl, butyl, isobutyl, tert-butyl, pentyl, hexyl, heptyl, octyl, or 2-methylpentyl.

The term “cycloalkyl” refers to saturated monocyclic, bicyclic, tricyclic, or other polycyclic hydrocarbon groups. Any atom can be substituted, e.g., by one or more substituents. Cycloalkyl groups can contain fused rings. Fused rings are rings that share a common carbon atom. Cycloalkyl moieties can include, e.g., cyclopropyl, cyclohexyl, methylcyclohexyl (the point of attachment to another moiety can be either the methyl group or a cyclohexyl ring carbon), cycloheptyl, adamantyl, and norbornyl.

The term “haloalkyl” refers to an alkyl group in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on an alkyl group can be replaced by more than one halogens (e.g., 2, 3, 4, 5, 6, 7, 8, 9, 10, 11, 12, 13, 14, 15, 16, 17, 18, 19, 20, 21, 22, 23, 24, 25, 26, etc. hydrogen atoms), which can be the same or different. “Haloalkyl” also includes alkyl moieties in which all hydrogens have been replaced by halo (e.g., perhaloalkyl, such as trifluoromethyl).

The term “aralkyl” refers to an alkyl moiety in which an alkyl hydrogen atom is replaced by an aryl group. Aralkyl includes groups in which more than one hydrogen atom on an alkyl moiety has been replaced by an aryl group. Any ring or chain atom can be substituted e.g., by one or more substituents. Examples of “aralkyl” include without limitation benzyl, 2-phenylethyl, 3-phenylpropyl, benzhydryl, and trityl groups.

The term “alkenyl” refers to a straight or branched hydrocarbon chain containing 2-20 carbon atoms and having one or more double bonds. Any atom can be substituted, e.g., by one or more substituents. Alkenyl groups can include, e.g., allyl, propenyl, 2-butenyl, 3-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted, e.g., by one or more substituents. Alkynyl groups can include, e.g., ethynyl, propargyl, 3-methylbutynyl, and 3-hexynyl. One of the triple bond carbons can optionally be the point of attachment of the alkynyl substituent.

The term “alkoxy” refers to an —O-alkyl radical. The term “haloalkoxy” refers to an —O-haloalkyl radical. The term aryloxy refers to an —O-aryl radical. The term “hydroxyalkyl” refers to an alkyl group which is substituted with one or more hydroxy groups. The nitrogen atom in an amino or amido group present in a steroid derivative of this invention can be mono- or di-substituted with an alkyl, a cycloalkyl, a heterocycloalkyl, an aryl, or a heteroaryl.

The term “heterocyclyl” refers to a monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The heteroatom can optionally be the point of attachment of the heterocyclyl substituent. Any atom can be substituted, e.g., by one or more substituents. The heterocyclyl groups can contain fused rings. Fused rings are rings that share a common carbon atom. Heterocyclyl groups can include, e.g., tetrahydrofuranyl, tetrahydropyranyl, piperidinyl, morpholino, pyrrolinyl, and pyrrolidinyl.

The term “cycloalkenyl” refers to partially unsaturated monocyclic, bicyclic, tricyclic, or other polycyclic hydrocarbon groups. The unsaturated carbon can optionally be the point of attachment of the cycloalkenyl substituent. Any atom can be substituted e.g., by one or more substituents. The cycloalkenyl groups can contain fused rings. Fused rings are rings that share a common carbon atom. Cycloalkenyl moieties can include, e.g., cyclopentenyl, cyclohexenyl, cyclohexadienyl, norbornyl, or cyclooctenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The unsaturated carbon or the heteroatom can optionally be the point of attachment of the heterocycloalkenyl substituent. Any atom can be substituted, e.g., by one or more substituents. The heterocycloalkenyl groups can contain fused rings. Fused rings are rings that share a common carbon atom. Heterocycloalkenyl groups can include, e.g., tetrahydropyridyl, and dihydropyranyl.

The term “aryl” refers to a monocyclic, bicyclic, or tricyclic aromatic moiety and can contain fused rings. Fused rings are rings that share a common carbon atom. Typical examples of aryl include phenyl, naphthyl, and anthracenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). Any atom can be substituted, e.g., by one or more substituents. Heteroaryl groups can contain fused rings. Fused rings are rings that share a common carbon atom. Heteroaryl groups include pyridyl, thienyl, furanyl, imidazolyl, and pyrrolyl.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on, e.g., an alkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituents on a group are independently any one single, or any subset of the aforementioned substituents. In another aspect, a substituent may itself be substituted with any one of the above substituents.

The positions of substituents on each of the cyclic groups described herein may be at any available position, unless specified otherwise. For example, a methyl substituent on a benzene ring can be attached at the ortho, meta, or para position.

For convenience, a divalent moiety is named herein as if it were a monovalent moiety. For example, “alkyl,” such as CH₃, which is assigned to, e.g., X, actually stands for “alkylene,” such as —CH₂—. As recognized by a skilled person in the art, steroid derivatives described herein contain stereocenters. Both the racemic mixtures of isomers and the optically pure isomers are within the scope of this invention.

The details of one or more embodiments of the invention are set forth in the accompanying drawings and the description below. Other features and advantages of the invention will be apparent from the description and from the claims.

### DETAILED DESCRIPTION

In some embodiments, the steroid derivatives of this invention can have formula (II):

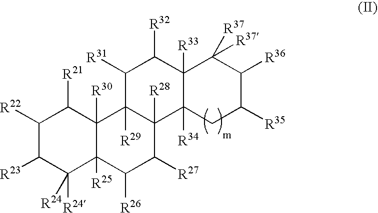

In some embodiments, each of R²¹, R²², R^{24′}, R³¹, and R^{37′}, independently of one another, can be hydrogen, halo, hydroxy, oxo, —O-sulfonic acid, C₁-C₁₂ (e.g., C₁, C₂, C₃, C₄, C₅, C₆, C₇, C₈, C₉, C₁₀, C₁₁ or C₁₂) alkyl, C₂-C₁₂ (e.g., C₂, C₃, C₄, C₅, C₆, C₇, C₈, C₉, C₁₀, C₁₁, or C₁₂) alkenyl, C₂-C₁₂ (e.g., C₂, C₃, C₄, C₅, C₆, C₇, C₈, C₉, C₁₀, C₁₁, C₁₂) alkynyl, NR^{a}R^{b}, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, —C(O)R^{c}, —C(O)OR^{c}, —OC(O)R^{c}, —C(O)NR^{a}R^{b}; or —NR^{d}C(O)R^{c}.

In some embodiments, each of R²³, R²⁴, R²⁶, R²⁷, R³², R³⁵, R³⁶, independently of one another, can be hydrogen, halo, hydroxy, oxo, —O-sulfonic acid, C₁-C₁₂ (e.g., C₁, C₂, C₃, C₄, C₅, C₆, C₇, C₈, C₉, C₁₀, C₁₁, or C₁₂) alkyl, C₂-C₁₂ (e.g., C₂, C₃, C₄, C₅, C₆, C₇, C₈, C₉, C₁₀, C₁₁, or C₁₂) alkenyl, C₂-C₁₂ (e.g., C₂, C₃, C₄, C₅, C₆, C₇, C₈, C₉, C₁₀, C₁₁, or C₁₂) alkynyl, NR^{a}R^{b}, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, —C(O)R^{c}, —C(O)OR^{c}, —OC(O)R^{c}, —C(O)NR^{a}R^{b}; or —NR^{d}C(O)R^{c}. In some embodiments, R²³ together with R²⁴ can be a bond, R²⁴ together with R²⁵ can be a bond, R²⁵ together with R²⁶ can be a bond, R²⁷ together with R²⁸ can be a bond, R³² together with R³³ can be a bond, or R³⁵ together with R³⁶ can be a bond.

In some embodiments, each of R²⁵, R²⁸, and R³³, independently of one another, can be hydrogen or C₁-C₁₂ (e.g., C₁, C₂, C₃, C₄, C₅, C₆, C₇, C₈, C₉, C₁₀, C₁₁, or C₁₂) alkyl. In some embodiments, R²⁵ together with R²⁴ can be a bond, R²⁵ together with R²⁶ can be a bond, R²⁸ together with R²⁷ can be a bond, or R³³ together with R³² can be a bond.

In some embodiments, each of R²⁹, R³⁰, and R³⁴, independently of one another, can be hydrogen or C₁-C₁₂ (e.g., C₁, C₂, C₃, C₄, C₅, C₆, C₇, C₈, C₉, C₁₀, C₁₁, or C₁₂) alkyl.

In some embodiments, R³⁷ can be C₁-C₂₀ (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkyl substituted with hydroxy, oxo, NR^{a}R^{b}, C₁-C₁₂ (e.g., C₁, C₂, C₃, C₄, C₅, C₆, C₇, C₈, C₉, C₁₀, C₁₁, or C₁₂) alkoxy, —C(O)R^{c}, —OC(O)R^{c}, or —NR^{d}C(O)R^{c}; or —C(O)NR^{a}R^{b}.

In some embodiments, each of R^{a}, R^{b}, and R^{d}, at each occurrence can be, independently of one another, hydrogen or C₁-C₁₀ (e.g., C₁, C₂, C₃, C₄, C₅, C₆, C₇, C₈, C₉, or C₁₀) alkyl.

In some embodiments, R^{c}, at each occurrence can be, independently, C₁-C₁₂ (e.g., C₁, C₂, C₃, C₄, C₅, C₆, C₇, C₈, C₉, C₁₀, C₁₁, or C₁₂) alkyl; C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl; heteroaralkyl including 6-20 (e.g., 6, 7, 8, 9, 10, 11, 12,13, 14, 15, 16, 17, 18, 19, or 20) atoms; C₃-C₁₆ (e.g., 3, 4, 5, 6, 7, 8, 9, 10, 11, 12, 13, 14, 15, or 16) cycloalkyl; C₃-C₁₆ (e.g., 3, 4, 5, 6, 7, 8, 9, 10, 11, 12, 13, 14, 15, or 16) cycloalkenyl; heterocyclyl including 3-16 (e.g., 3, 4, 5, 6, 7, 8, 9, 10, 11, 12, 13, 14, 15, or 16) atoms; heterocycloalkenyl including 3-16 (e.g., 3, 4, 5, 6, 7, 8, 9, 10, 11, 12, 13, 14, 15, or 16) atoms; C₆-C₁₆ (e.g., C₆, C₇, C₈, C₉, C₁₀, C₁₁, C₁₂, C₁₃, C₁₄, C₁₅, or C₁₆) aryl; or heteroaryl including 5-16 (e.g., 5, 6, 7, 8, 9, 10, 11, 12, 13, 14, 15, or 16) atoms.

m can be 0, 1,or2.

In all embodiments with respect to compounds having formula (II), at least one of R²³ and R²⁴, R²⁴ and R²⁵, R²⁵ and R²⁶, R²⁷ and R²⁸, R³² or and R³³, or R³⁵ and R³⁶, together is a bond.

For ease of exposition, it is understood that any recitation of ranges (e.g., C₁-C₂₀) or subranges of a particular range (e.g., C₁-C₁₂) for any substituent defined herein, e.g., R¹, R², R³, etc. expressly includes each of the individual values that fall within the recited range, including the upper and lower limits of the recited range. For example, the range C₁-C₄ alkyl is understood to mean (e.g., C₁, C₂, C₃, or C₄) alkyl.

A subset of steroid derivatives having formula (II) can include those in which R²⁵ together with R²⁶ is a bond and m is 0. In some embodiments, the steroid derivative can be a cholesterol derivative. In certain embodiments, R³⁷ can be C₁-C₂₀ alkyl substituted with hydroxy. When the carbon bearing the hydroxy group is a stereogenic center, the stereogenic center can have either the R or S configuration. Exemplary compounds of formula (II) include 22(R)-hydroxycholesterol and 24(S)-hydroxycholesterol:

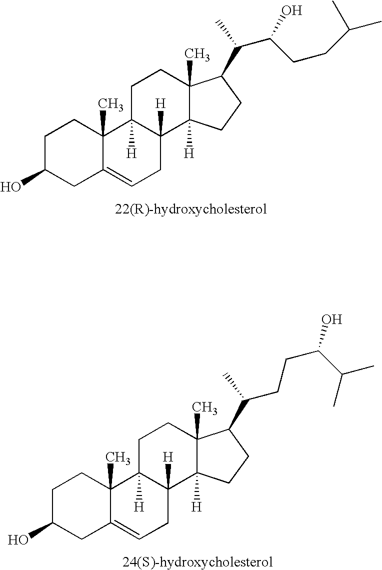

The compounds described herein an be obtained from commercial sources (e.g., 22(R)-hydroxycholesterol and 24(S)-hydroxycholesterol can be obtained from Steraloids (Newport, R.I.)), or synthesized according to methods described herein and/or by conventional, organic chemical synthesis methods from commercially available starting materials and reagents.

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, steroid derivatives having formula (I) can be prepared by forming an amide bond between a steroid having a C17 carboxyl-containing substituent and an amino-containing compound or between a steroid having a C17 amino-containing substituent and a carboxyl-containing compound. Similarly, an ester bond can be formed between a steroid with a C17 carboxyl-containing substituent and a hydroxyl-containing compound, or between a steroid with a C17 hydroxyl-containing substituent and a carboxyl-containing compound. Some examples of a steroid that can be used as a starting material are cholic acid (e.g., ursodeoxycholic acid, hyocholic acid, and hyodeoxycholic acid), androstan-17-carboxylic acid (e.g., androstan-3-oxo-17-carboxylic acid and d5-androsten-3-ol-17-carboxylic acid) and pregnan-20-ol (e.g., d5-pregnan-3,17-diol or pregnan-17-ol-3-one). Synthesis of these steroids can be found in the literature, e.g., Roda A. et al., F. Lipid Res. vol. 35, pages 2268-2279 (1994) and Roda A. et al., Dig. Dis. Sci. vol. 34, pages 24S-35S (1987). Some examples of compounds that can be used to couple to a steroid to form a steroid derivative of this invention are aniline, glycine, phenylalanine, or benzoic acid. Examples of a coupling reagent that can be used in the amide- or ester-forming reaction include 1-ethyl-3-[3-dimethylaminopropyl]-carbodiimide (EDC), dicyclohexyl-carbodiimide (DCC), N-hydroxybenzo-triazole (HOBt), 2-(1H-benzotriazole-1-yl)-1,1,3,3 -tetramethyluronium hexafluoro-phosphate (HBTU), or benzotriazole-1-yl-oxy-tris-pyrrolidino-phosphonium hexafluorophosphate (PyBOP). The amide- or ester-forming reaction can take place in any solvents that are suitable with the starting materials and reagents. Note that if the reaction takes place in an aqueous solvent, e.g., a buffered solution (or in combination with other miscible organic solvents such as alcohol), isolation of the steroid product for in vitro or in vivo screening assays is not necessary, as the product is already in suitable assaying conditions, i.e., in an aqueous buffered medium. Protection of functional groups, e.g., hydroxyl or keto, on the steroids is not needed. See, e.g., Example 1 below. Due to the simplicity of the reaction, it can be easily automated. Isolation and quantification of the product can be done by thin-layer chromatography, high pressure liquid chromatography, gas chromatography, capillary electrophoresis, or other analytical and preparative procedures. Trifluoromethyl- and taurine-conjugated steroid derivatives can be prepared according to methods described in Li, S. et al., Chem. Phys. Lipids 99:33-71 (1999) and Kurosawa, T. et al., Steroids, 60:439-444 (1995), respectively. As to the preparation of 3β-hydroxy-5-cholesten-25(R)-26-carboxylic acid derivatives, see Kim, H. et al., J. Lipid Res. 30:247 (1989) and Varma, R. K. et al., J. Org. Chem. 40:3680 (1975). Steroid derivatives having a side chain that contains a double bond, e.g., between C24 and C25, can be prepared according to the following scheme:

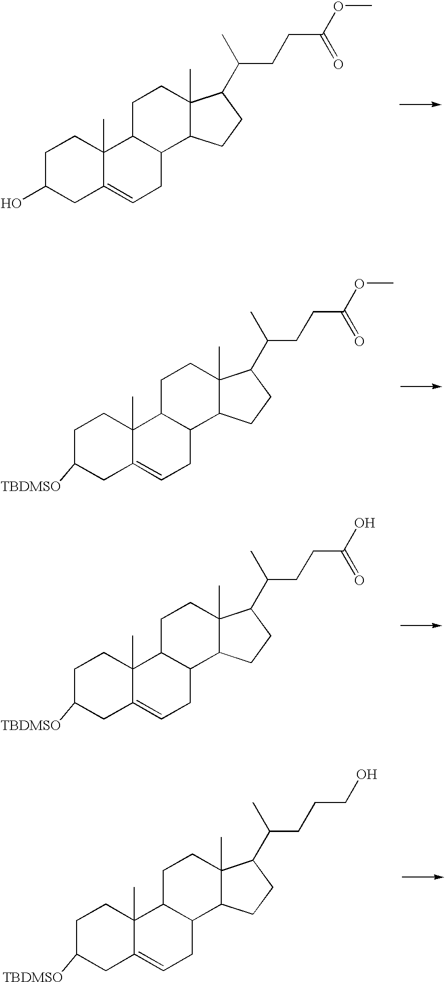

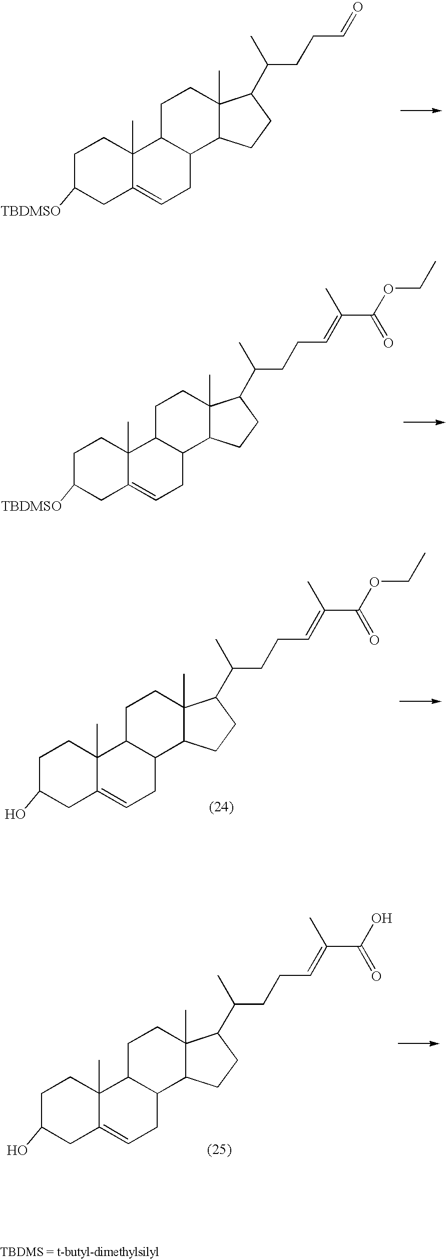

3-beta-t-butyldimethylsilyloxy-delta[5]-cholen-24-al and 3-alpha,6-alpha-di(t-butyldimethylsilyloxy)5-beta-cholan-24-al were prepared using NaBH4 and pyridinium chlorochromate according to methods described in Somanathan et al., Steroids 43:651-655 (1984). Ethyl-3-beta-t-butyldimethylsilyloxy-delta[5,24]-cholestenoate and ethyl-3a,6a-di(t-butyldimethylsilyloxy)-delta[24]-cholestanoate were then prepared via Wittig-Horner reaction using triethyl 2-phosphono-propionate and a suitable base according to methods described in Lund et al., Arterioscler. Thromb. Vasc. Biol. 16:208-212 (1996). After the t-butyldimethylsilyloxyl groups were removed, ethyl ester groups were hydrolyzed under alkaline conditions.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In some embodiments, the steroid derivatives described herein, e.g., Liver X receptor agonists having formula (I) or (II), can be used for treating cancer, e.g., a sex hormone-dependent cancer (e.g., prostate cancer or breast cancer).

In some embodiments, the sex hormone-dependent cancer can be prostate cancer. In certain embodiments, the prostate cancer can be an androgen-dependent prostate cancer. In certain embodiments, the prostate cancer can be resistant to conventional androgen deprivation and/or antiandrogen therapies (e.g., an androgen-independent prostate cancer, e.g., a hormone-refractory prostate cancer). For example, a subject (e.g., a patient, e.g., a human patient) can have at least one prostate cancer tumor that is relatively resistant to androgen deprivation and/or antiandrogen therapies, e.g., an androgen-independent prostate cancer tumor. In some embodiments, the subject can further be substantially free of androgen-dependent prostate cancer tumors. Androgen-independent prostate cancer and hormone-refractory prostate cancer are described in, e.g., Kasamon, et al., Curr Opin. Urol. 14: 185-193 (2004).

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of any of the formulae described herein). Alternatively, those agents may be part of a single dosage form, mixed together with the compounds of this invention in a single composition (e.g., simultaneously or at about the same with one or more compounds of any of the formulae described herein). When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the therapeutic agent can be an RXR agonist (e.g., LGD1069, Bexarotene, Tagretin). RXR agonists are described in, e.g., Lippman et al., Journal of Nutrition (2000) Supplement 479S-482S; and Staels J. Am. Acad. Dermatol. (2001) 45, S158-S167.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 2000 mg/Kg, (e.g., from about 0.01 mg/Kg to about 100 mg/kg, from about 0.1 mg/Kg to about 100 mg/Kg, 1 mg/kg to about 2000 mg/Kg, from about 1 mg/Kg to about 1000 mg/Kg, or from about 1 mg/kg to about 500 mg/kg; from about 1 mg/Kg to about 100 mg/Kg, from about 1 mg/Kg to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable excipients, carriers, adjuvants or vehicles. In some cases, the pH of the formulation may be adjusted with pharmaceutically acceptable acids, bases or buffers to enhance the stability of the formulated compound or its delivery form. In some embodiments, solubilizing agents such as cyclodextrins, or other solubilizing agents well-known to those familiar with the art, can be utilized as pharmaceutical excipients for delivery of the therapeutic compounds.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution, isotonic sodium chloride solution, and 5% glusoce. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil, sesame oil or castor oil, e.g., in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, gel seal, capsules, tablets, syrups, emulsions and aqueous suspensions, dispersions and solutions. In the case of tablets for oral use, carriers which are commonly used include starch, sugar bentonite, lactose and corn starch. Lubricating agents, such as magnesium stearate, are also typically added. Tablets may be formulated in accordance with the conventional procedure by compressing mixtures of the compound of this invention and a solid carrier, and a lubricant. The compounds of this invention can also be administered in a form of a hard shell tablet or a capsule containing a binder (e.g., lactose or mannitol) and a conventional filler. For oral administration in a capsule form, useful diluents include gelatin, cellulose derivatives, lactose and dried corn starch. When aqueous suspensions and/or emulsions are administered orally, the active ingredient may be suspended or dissolved in an oily phase is combined with emulsifying and/or suspending agents. In some embodiments, the vehicle for oral administration can be a pharmaceutically-acceptable oils, e.g., a natural oil, such as olive oil, sesame oil or castor oil. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The level of interaction between the UR or LXRa protein and a steroid derivative of this invention can be preliminarily evaluated using various assays as described below:

Protease protection assay is a simple assay for measuring the level of interaction between a test steroid and the UR or LXRa protein. This assay can be done by using a ³⁵S-Met radiolabeled rat UR or human LXRa protein. The radiolabeled protein is then incubated with the steroid of this invention and digested with a protease, e.g., trypsin. A control experiment is done by incubating UR receptor with a protease but without the steroid. Protein fragments from both assays are electrophoresed on a polyacrylamide gel. The fragments from each of the assays can be visualized by exposing the gel to X-ray films and compared side-by-side. A test steroid, if binds to the UR or LXRa protein, will protect the receptor from being digested by the protease. As a result, reactions that result in binding between the steroid and UR will lead to fewer bands of low molecular weights than those that do not result in binding between the two molecules.

The co-activator binding assay employs a fusion protein formed between a glutathione S-transferase (GST) and a co-activator of UR, e.g., Grip1. The GST moiety of the fusion protein binds to a glutathione-coated solid support, thereby immobilizing the fusion protein. UR and a steroid of this invention are then incubated with the immobilized fusion protein. Subsequently, any bound UR is released and collected from the solid support. The proteins are then electrophoresed on a polyacrylamide gel and visualized by exposing the gel to X-ray films. If the steroid interacts with UR, less UR will bind to the fusion protein, and a lighter band would therefore result on the gel. By monitoring the intensity of the band of the bound UR, one can estimate the binding of the steroid to UR.

Yeast two-hybrid binding assay is a sensitive assay for identifying UR modulating compounds by monitoring transcriptional activation. General descriptions of these assay can be found in, e.g., Chien C. T. et al., Proc. Natl. Acad. Sci. USA, vol. 88, 9578-9582 (1991); Fields, S. et al., Nature, vol. 340, 245-247 (1989); and Green, M. B. et al., Curr. Biol., vol. 2, 403-405 (1992). In this screening method, a steroid of this invention that modulates the interaction of UR or LXRa with its natural ligand will have an effect on the transcriptional activation of a reporter gene. In a specific assay, two plasmids are introduced into a yeast cell. One expresses a fusion protein having a GAL4 DNA binding domain and a UR natural ligand, and the other expresses a fusion protein containing a UR ligand binding domain and a GAL4 activation domain. If the steroid interacts with UR and disrupts the binding of UR to its natural ligand, the activity of the reporting gene (Gal4) will be altered. The changes in reporter activities (i.e., β-galactosidase activities) can be measured with a commercial luminescence kit.

Mammalian cell transfection can also be used to screen steroid derivatives that affect the interaction between the UR protein and a steroid of this invention. A rat UR or human LXR gene and a human RXRa gene are cloned into a mammalian expression vector (e.g., pSG5 from Strategene) and overexpressed. A heterologous promoter is formed by inserting four tandem repeats of a hormone response element DR4 into the vector upstream to a c-fos promoter sequence, which is followed by a sequence encoding luciferase. The entire construct is named DR4-fos-luc. DR4-fos-luc is then co-transfected with pSG5/rUR or CMV/hLXR and pSG5/hRXRa into mammalian cells, e.g., COS-1 cells. An ethanol solution containing a steroid of this invention is then added to the transfected cells. The steroid, if interacts to the UR or LXRa protein, affects the level at which the luciferase gene is activated. The cells are then lysed and assayed for luciferase activity with a commercial assay kit and a luminometer. A high intensity of luminescence indicates that the steroid is a potent UR or LXR agonist.

Another chimeric receptor that can be used in this assay is constructed by fusing oligonucleotides encoding the ligand-binding domain of rat UR to a human AR gene lacking ligand-binding site coding region. For this chimeric receptor, a reporter gene ARE-fos-luc is constructed by inserting three tandem repeats of Androgen Response Element (ARE) into the vector upstream to a c-fos promoter which is followed by a luciferase reporter gene. After adding a steroid of this invention to the medium of the transfected cells, the steroid can interact with UR and affect the level of activation of ARE-fos-luc in cultured cells. The level of luminescence activity thus indicates the level of UR modulation by the steroid.

Yet another assay involves expressing rUR gene in PC-3 cells by retroviral infection. See Underwood et al.,J. Biol. Chem., vol. 273, pages 4266-4274 (1998). The transfected cells are then seeded in media containing delipidated serum and then treated with a solution containing a steroid of this invention. The PC-3 cells are later washed with phosphate buffered saline (PBS) and treated with 100 mg/ml amphotericin B in DMEM media without serum at 37EC. Amphotericin B functions to kill cells containing cholesterol in the cell membrane. The cells are then fixed in 10% TCA and stained with Sulforhodamine B after more washing. Viable cells are stained and can then be assessed using a colorimetric assay. The amount of dye is directly proportional to number of surviving cells on the culture plates. From comparing the number of viable cells between assays with and without a steroid, one can estimate the effect the steroid has on the de novo synthesis of cholesterol.

A still further assay makes use of nitrogen monoxide (NO) as an indicator of the level of inflammation. Cells from a murine macrophage cell line RAW264.7 are incubated with a steroid of this invention for 24 hours. The macrophages are then activated by adding lipopolysaccharide (LPS) and gamma-interferon. The NO production of activated macrophages can be monitored indirectly by quantifying NO2 in the media according to Green L. et al., Anal. Biochem., vol. 126, 131-138 (1982). The reduced amount of NO in comparison to that of a control experiment in which no steroid is used indicates that the steroid used in the assay has inhibitory effect on inflammation.

Using the same murine macrophage cell line RAW264.7, constitutive expression of rat UR and human RXRa gene by retroviral systems transforms these cells into foam-cell-like morphology and integrated into clumps while increasing cell sizes and undergo apoptosis. Foam cells originated from macrophages are the major components in pathological plaque which is usually found on the inner wall of blood vessels in patients suffering from atherosclerosis. Steroid derivatives of this invention which modulate UR can suppress the progression of macrophage-foam cell transformation at different stages, and can be used in the treatment or prevention of atherosclerosis. See Kellner-Weibel et al., Arterioscler. Thromb. Vasc. Biol., vol. 18, pages 423-431 (1998).

Yet another assay measures the effect of a steroid of this invention has on the level of adipocyte differentiation on fibroblasts. Specifically, the level of adipocyte differentiation in murine fibroblasts 3T3-L1 containing rat UR gene at sub-confluent conditions is measured. Constitutive expression of rat UR gene in murine fibroblasts 3T3-L1 can be done by using retroviral systems. Full-length rat UR cDNA are inserted into retroviral expression vector MV7. Infected 3T3-L1 cells that are G418-resistant are treated with insulin, dexamethacine, and 1-methyl-3-isobutylxanthine (MIX) to induce adipocyte differentiation. A control experiment can be done by inserting human UR cDNA into MV7 in the antisense orientation. Cells infected with hUR-antisense constructs and parent 3T3-L1 cells are also treated with the same insulin cocktail under same cell density. Cells infected with rUR are shown to accumulate more Red oil O positive lipid drops than parent cells, while cells infected with hUR antisense are shown to have less Red oil O positive lipid drops. Thus, the finding shows that the expression of UR in fibroblasts plays a role in adipocyte differentiation.

Without further elaboration, it is believed that one skilled in the art can, based on the description herein, utilize the present invention to its fullest extent. The following specific examples, which described syntheses, screenings, and biological testings of various compounds of this invention, are therefore, to be construed as merely illustrative, and not limitative of the remainder of the disclosure in any way whatsoever. All publications recited herein, including patents, are hereby incorporated by reference in their entirety.

### Preparation of phenylalanine Conjugated-Steroid Derivatives

To a stirred solution of L-(or D-) phenylalanine ester hydrochloride (2 mmol) in dry DMF (10 mL) was added triethylamine (2 mmol) and the mixture was stirred at room temperature for 10 minutes. Bile acid (1 mmol) and 1-ethyl-3-[3-dimethylaminopropyl]-carbodiimide (2 mmol) were then added and the suspension was stirred at room temperature overnight. The reaction mixture was diluted with water and ethyl acetate. The organic layer was separated and the water layer was extracted with ethyl acetate again. The combined organic layer was then washed with 1N HCl, water, 1N NaOH and water, and dried (MgSO₄). The solvent was removed under reduced pressure to afford the steroid derivatives which were then analyzed by Thin Layer Chromatography, High Pressure Liquid Chromatography, and/or proton-NMR.

### Preparation of ethyl-3-alpha,6-alpha-dihydroxy-delta[24]-5-beta-cholestanoate

Ethyl-3-alpha,6-alpha-dihydroxy-delta[24]-5-beta-cholestanoate was prepared according to methods described above. ¹H NMR: 0.63 (C18); 0.90 (C19); 1.29 (C21); 1.88 (C26); 3.61 (C3); 4.04 (C6); 4.22 (C28); 5.88 (C24).

### Preparation of 3-alpha,6-alpha-dihydroxy-delta[24]-5-beta-cholestan-27-oic acid

3-Alpha,6-alpha-dihydroxy-delta[24]-5-beta-cholestan-27-oic acid was prepared according to methods described above. ¹H NMR: 0.63 (C18); 0.90 (C19); 1.29 (C21); 1.88 (C26); 3.61 (C3); 4.04 (C6); 4.22 (C28); 6.85 (C24).

### Preparation of ethyl-3-beta-hydroxy-delta[5,24]-cholestenoate

Ethyl-3-alpha,6-alpha-dihydroxy-delta[24]-5-beta-cholestanoate was prepared according to methods described above. ¹H NMR: 0.68 (C18); 0.95, 1.00 (C19, C21); 1.83 (C26); 3.50 (C3); 4.19 (C28); 5.34 (C5); 6.74 (C24); ¹³C NMR: 72.0 (C3); 121.9 (C5); 143.3 (C6); 168.8 (C27); 127.8, 141.2, 144.0 (C24, C25).

### Preparation of 3-beta-hydroxy-delta[5,24]-cholesten-27-oic acid

3-Alpha,6-alpha-dihydroxy-delta[24]-5-beta-cholestan-27-oic acid was prepared according to methods described above. ¹H NMR: 0.68 (C18); 0.95, 1.00 (C19, C21); 1.83 (C26); 3.50 (C3); 4.19 (C28); 5.34 (C5); 6.79 (C24).

Yeast Two-Hybrid Binding Assay

A commercial yeast two-hybrid kit from Stratagene, HybriZAP-2.1™, was used to construct primary screening system. Four pairs of degenerated oligonucleotides were annealed, digested with EcoRI and SalI, and purified. The sequences of the four pairs of oligonucleotides are listed as follows (N represents A, G, T or C):

| | | |
|---|---|---|
| WB1: | 5′-GTA TCG CCG GAA TTC NNN TTG | (SEQ ID NO:2) |
| | NNN NNN TTG TTG NNN NNN TAA | |
| | GTC GAC TCT AGA GCC-3′ | |
| WB2: | 5′-GGC TCT AGA GTC GAC TTA NNN | (SEQ ID NO:3) |
| | NNN CAA CAA NNN NNN CAA NNN | |
| | GAA TTC CGG CGA TAC-3′ | |
| LS1: | 5′-GTA TCG CCG GAA TTC ATC TTG | (SEQ ID NO:4) |
| | CAC AGA TTG TTG CAA GAA TAA | |
| | GTC GAC TCT AGA GCC-3′ | |
| LS2: | 5′-GGC TCT AGA GTC GAC TTA TTC | (SEQ ID NO:5) |
| | TTG CAA CAA TCT GTG CAA GAT | |
| | GAA TTC CGG CGA TAC-3′ | |
| WD1: | 5′-GTA TCG CCG GAA TTC NNN TTG | (SEQ ID NO:6) |
| | NNN NNN TGG TTG TTG NNN NNN | |
| | TAA GTC GAC TCT AGA GCC-3′ | |
| WD2: | 5′-GGC TCT AGA GTC GAC TTA NNN | (SEQ ID NO:7) |
| | NNN CAA CAA CCA NNN NNN CAA | |
| | NNN GAA TTC CGG CGA TAC-3′ | |

The purified fragments were cloned into the yeast vector pBD-GAL4 (Strategene) of the same restriction sites. The resulting plasmid pCAM/BDs expressed a fusion protein with a GAL4 DNA-binding domain (amino acid 1-147 of Gal4) and a polypeptide of ten amino acid in length with a LXXLL (SEQ ID NO: 8) or LXXWLL (SEQ ID NO: 9) motif. UR ligand binding domain (amino acids 141 to 443 of rUR) was generated with PCR and inserted into another yeast vector pAD-GAL4-2.1 (Strategene) with cloning site EcoRI and XhoI. The resulting plasmid, p2.1/rURLB, expressed a fusion protein containing a Gal4 transcription activation domain (amino acids 761-881 of Gal4) and a rUR ligand binding domain.

Plasmids pCAM/BDs and p2.1/rURLB were co-transformed into an appropriate yeast strain by using lithium acetate and polyethylene glycol. The yeast was then grown on selective medium until the formed yeast colonies reached 2 mm. Colonies were picked and grown in selective medium for 15 hours at 30° C. and β-galactosidase activities were measured with a commercial luminescence kit.

Mammalian Cell Transfection Assay (1)

Rat UR and human RXRa gene were cloned into a mammalian expression vector pSG5 (Strategene) by transfection with calcium phosphate and overexpressed in cultured cells. A heterogeneous promoter was constructed by inserting into the vector four tandem repeats of DR4 with sequence 5′-TTC AGG TCA CAG GAG GTC AGA GAG CT-3′ (SEQ ID NO: 10) upstream to a c-fos promoter sequence (−56-+109) which was followed by a sequence encoding luciferase. The entire construct was named DR4-fos-luc. DR4-fos-luc was then co-transfected with pSG5/rUR and pSG5/hRXRa into COS-1 cells. 16-24 hours after transfection, a steroid derivative in ethanol was added to the medium until the maximum final concentration is 2 μM. The final concentration of solvent ethanol is 0.2%. After 24-48 hours, cells that were treated with the steroid were lysed and assayed for luciferase activity with a commercial assay kit and a luminometer.

A wide variety of compounds of this invention were tested and found to modulate transactivation activity of UR or LXRa. For example, steroid (1) (see page 5, supra), unexpectedly increased the luciferase activity by 15-fold in comparison to absence of steroid only for UR but not LXRa; steroid (2) unexpectedly increased the luciferase activity by 60-fold in comparison to absence of steroid only for LXRα but not UR; steroid (3), (5) or (10) can activate both UR or LXRa; steroid (7), (8), or (9) can antagonize UR or LXRα transactivation acitvity.

Mammalian Cell Transfection Assay (2)

In a similar fashion to the experiment described above, another chimeric receptor was constructed by fusing oligonucleotides encoding the ligand-binding domain of rat UR (141 to 443 amino acid residues) to a human AR gene lacking ligand-binding coding region (human AR 1 to 623 amino acid residues) and overexpressed in cultured cells. For this chimeric receptor, a reporter gene ARE-fos-luc was constructed by inserting into the vector three tandem repeats of Androgen Response Element (ARE) with a sequence 5′-TCG AGT CTG GTA CAG GGT GTT CTT TTG-3′ (SEQ ID NO: 11) upstream to a c-fos promoter sequence (−56-+109) which was followed by a sequence encoding luciferase.

Various steroid derivatives of this invention were found to modulate UR transactivation activity on DR4-fos-luc expression in the cultured cells. For example, steroid derivative (6) (see page 6, supra) unexpectedly increased the luciferase activity by 5-fold in comparison to the steroid starting material.

Mammalian Cell Transfection Assay (3)

Human embryonic kidney 293 cells were seeded into 48-well culture plates at 105 cells per well in DMEM supplemented with 10% fetal bovine serum. After 24 hours, cells were transfected by a calcium phosphate coprecipitation method with 250 ng of the pGL3/UREluc reporter gene which consists of three copies of AGGTCAagccAGGTCA fused to nucleotides −56 to +109 of the human c-fos promoter in front of the firefly luciferase gene in the plasmid basic pGL3 (Promega), 40 ng pSG5/hRXRa, 40 ng pSG5/rUR or CMX/hLXR, 10 ng pSG5/hGrip1, 0.4 ng CMV/R-luc (transfection normalization reporter, Promega) and 250 ng carrier DNA per well. Alternatively, 500 ng of the pGL2/7aluc reporter gene which consists of a single copy of nucleotides −101 to −49 of the rat 7a-hydroxylase gene fused to the SV40 promoter in front of the firefly luciferase gene in the plasmid basic pGL2 (Promega) was used instead of pGL3/UREluc. This reporter does not have response elements for COUP-TFII or HNF4. In some experiments, 500 ng of the human 7α-hydroxylase gene reporter, PH/hCYP7A-135, which consists of a single copy of nucleotides −135 to +24 of the human CYP7A gene fused to the firefly luciferase gene in the plamid basic pGL3 (Promega), was used instead of pGL2/7aluc. After another 12-24 hours, cells were washed with PBS and refed with DMEM supplemented with 4% delipidated fetal bovine serum. Steroid derivatives dissolved in ethanol were added in duplicate to the medium so that the final concentration of alcohol was 0.2%. After 24-48 hours, cells were harvested and luciferase activity was measured with a commercial kit (Promega Dual luciferase II) on a Monolight luminometer (Beckton Dickenson). Both LXR and UR form heterodimers with RXR for gene transactivation. The ligand for RXR, 9-cis retinoic acid, is known to activate the LXR/RXR heterodimer but addition of 9-cis retinoic acid to transactivation assays did not change the potency of either Δ⁵ or 6α-hydroxy steroids for activation of LXR or UR (data not shown). The expression of endogenous LXR and UR (and TR which also binds to a DR4 response element) were apparently low since reporter activation was low in the absence of added expression vectors for LXR or UR. Reporter activation was also low when the DR4 response-element was replaced with a glucocorticoid receptor response element. Each experiment was repeated as least twice to demonstrate reproducability. Relative light units were about 2×10⁷ for pGL3/UREluc, 1×10⁶ for pGL2/7aluc, 5×10⁴ for PH/hCYP7A-135 and 5×10⁵ for CMV/R-luc. Purity of synthesized steroid derivatives was verified by thin layer chromatography and structures were confirmed using proton and C¹³ magnetic resonance spectrometry. 3-Oxo-6α-hydroxy-5β-cholanoic acid methyl ester, 3α,6α-dihydroxy-5β-cholanoic acid methyl ester, and 3α,6α,7α-trihydroxy-5β-cholanoic acid methyl ester were found to be as potent as 3β-hydroxy-Δ⁵-cholanoic acid methyl ester as activators for LXR, with ED₅₀'s of about 150 nM. Loss of activity was seen when the 6α-hydroxy group was changed to a 6β configuration. In contrast to activity with LXR, 3β-hydroxy-Δ⁵-cholanoic acid methyl ester (ED₅₀ of 130 nM) was more active than 3-oxo-6α-hydroxy-cholanoic acid methyl ester (ED₅₀ of 550 nM) and 3α,6α-dihydroxy-cholanoic acid methyl ester (ED₅₀ of 500 nM) for UR activation.

Using the same assay, ED₅₀'s of 6α-hydroxylated steroids with 24-keto side chains include free and conjugated 3α,6α-dihydroxy-5β-cholanoic acid and 3α,6α,7α-trihydroxy-5β-cholanoic acid were determined. These steroid derivatives were found to be more selective activators of LXR than UR. 3α,6α-dihydroxy-5β-cholanoic acid activated LXR with an ED₅₀ of 17 mM for the free acid and 3 mM for its taurine conjugate. Free and taurine-conjugated 3α,6α-dihydroxy-5β-cholanoic acids activated UR with ED₅₀ of 55 mM and 11 mM, values three to four times higher than those for LXR. Cholanoic acid derivatives containing trifluoromethyl moiety were also found to be selective activators of LXR.

The ability of taurine-conjugated 3α,6α-dihydroxy-5β-cholanoic acid to activate LXR using the natural response element derived from the rat 7a-hydroxylase promoter was also investigated. It was found that taurine-conjugated 3α,6α-dihydroxy-5β-cholanoic acid activated LXR but not UR using this reporter gene, with an ED₅₀ of 10 mM. To investigate if LXR can activate human CYP7A gene transcription, a chimeric reporter plasmid, in which the nucleotides −135 to +24 of the human CYP7A promoter were fused to the luciferase gene, was used in a co-transfection assay in human embryonic kidney 293 cells along with LXR, RXR and Grip1 expression plasmids. It was found that LXR can activate reporter gene expression in the presence of taurine-conjugated 3α,6α-dihydroxy-5β-cholanoic acid. Taurine-conjugated 3α,7α-dihydroxy-5β-cholanoic acid, on the other hand, suppressed reporter gene expression. Another compound, 3β-hydroxy-5-cholesten-25(R)-26-carboxylic acid activated LXR with an ED₅₀ of 300 nM and UR with an ED₅₀ of over 2 μM. Its taurine-conjugated counterpart was also found to be able to transactivate both LXR and UR. On the other hand, many of its related metabolites were found to be inactive on either receptors.

Protease Protection Assay

Rat UR protein radio-labeled with ³⁵S-Met is produced with a commercial kit in an in vitro system. The radio-labeled protein is incubated with steroid derivatives with final concentration of up to 1 mM for 2 hours on ice, and digested with trypsin for 30 minutes at 37° C. for 20 minutes. The protected fragments were separated from free ³⁵S-Met by polyacrylamide electrophoresis and visualized by exposing dried gels to X-ray films.

The patterns of the X-ray film indicate that steroid derivatives of this invention bind to and protect UR from being digested by trypsin. Some examples of such a steroid derivative include 5β-androstan-3a, 17b-diol, 5β-androstan-3a-ol-16-one, Δ⁵-Pregnen-3b-ol20-one, 5a-androstan-3-one, 5α-androstan-17-ol-3-one, 5a-androstan-3b-ol-17-carboxylic acid, 5a-pregnan-3,20-dione, and Δ⁵-androsten-3b, 17b-diol.

Incubation of UR with increasing concentrations of trypsin in the absence of 3α,6α-dihydroxy-5β-cholanoic acid methyl ester leads to extensive digestion of the receptor. In contrast, when UR was incubated with 5 mM 3α,6α-dihydroxy-5β-cholanoic acid methyl ester, two protease-resistant fragments of 35 and 26 kDa were observed. A similar protected pattern was observed with taurine-conjugated 3α,6α-dihydroxy-5β-cholanoic acid.

Co-Activator Binding Assay

A fusion protein formed between glutathione S-transferase and Grip1 (termed GST-Grip1) was expressed in E. Coli. The bacteria was lysed by sonication in the presence of detergent NP40 0.1% and Tween-20 0.5%. Soluble GST-Grip1 was separated from insoluble debris by centrifugation at 50,000 G at 4° C. for 30 minutes. The soluble fusion protein was then immobilized to glutathione-agarose. Radiolabeled rat UR protein was incubated with GST-Grip1 coated glutathione-agarose in the presence of a test compound of this invention for 2 hours at 22° C. under agitation. UR that did not bind to the agarose was washed away. Bound UR was eluted with solution containing SDS and β-mercaptoethanol and separated from free ³⁵S-Met with polyacrylamide electrophoresis, and finally visualized by exposure the dried gel to X-ray films. Diosgenin was shown to be capable of promoting UR and Grip 1 protein interaction in this assay.

Another fusion protein GST-rUR was expressed in E. Coli strain BL21 using the expression plasmid pGEX using a method similar to that as described above. Transfected cells were lysed by one cycle of freeze-thaw and sonication. Supernatant, prepared by centrifugation at 45,000 G for 1 hour, was incubated with glutathione-agarose for 10 min at 4° C. The agarose was washed with binding buffer (20 mM Hepes, pH7.5, 10 mM EDTA, 10 mM Na₂MoO₄, 1 mM β-mercaptoethanol, 1 mM DTT, 0.5 mM PMSF, 2 ug/ml aprotinin). Human Grip1 was produced by in vitro translation using a rabbit reticulocyte lysate and labeled with [³⁵S]-methionine. [⁵]-Grip1 in reticulate lysate (2 ml) was added to GST-UR bound to agarose beads in 100 ul binding buffer. Test chemicals in ethanol were added to the mixture and the slurry was shaken at room temperature for 30 min. The agarose beads were then washed three times with binding buffer. Bound protein was eluted with SDS-PAGE loading buffer and separated on a 8% SDS-PAGE gel. Gels were dried and subjected to autoradiography. Radioactive Grip1 was measured with a STORM phosphoimager (Molecular Dynamics).

Both 3α,6α-dihydroxy-5β-cholanoic acid methyl ester and 22R-hydroxy cholesterol promoted interaction of Grip1 with GST-UR and taurine-conjugated 3α,6α-dihydroxy-5β-cholanoic acid promoted interaction of Grip1 with GST-LXR. Taurine-conjugated 3α-hydroxy-5β-cholanoic acid, taurine-conjugated 3α-hydroxy-5β-cholanoic acid, and taurine-conjugated 3α,7α-dihydroxy-5β-cholanoic acid all failed to enhance coactivator-receptor interaction under the same conditions.

Using the same conditions, 3β-hydroxy-5-cholesten-25(R)-26-carboxylic acid was found to bind to and form complexes with LXR and nuclear receptor co-activator Grip 1, indicating that this acid bound to LXR and induced a conformation change that favored co-activator binding. In a dose response analysis, 3β-hydroxy-5-cholesten-25(R)-26-carboxylic acid increased the amount of [³⁵S]-Grip1 bound to LXR with an EC₅₀ value of 300 nM, which correlates with the cell-based transfection assay. These data showed that 3β-hydroxy-5-cholesten-25(R)-26-carboxylic acid is a LXR agonist.

Inhibition of de novo Cholesterol Synthesis in Cultured Cells

On day 1, PC-3 cells stably expressing rUR gene by retroviral infection were seeded in media containing delipidated serum. On day 2, cells were treated with an ethanol solution containing a test compound at maximum concentration of 2 μM. On day 3, cells were washed with PBS and treated with 100 mg/ml amphotericin B in Dulbecco's Modified Eagle Medium (DMEM) without serum at 37□C. 4 hours later, cells were then washed and treated with solution containing 80% water and 20% DMEM for 30 minutes. Surviving cells were assessed using a colorimetric assay. Cells were fixed in 10% trichloroacetic acid (TCA) and stained with sulforhodamine B. The amount of dye is linear to number of fixed cells on the culture plates. Cells with cholesterol in the cell membrane were killed by amphotericin B treatment.

Compounds of this invention were found to inhibit cholesterol synthesis of the cell to various extent.

Measuring the Level of Inflammation in Cells by Monitoring the Amount of NO₂

Murine macrophage cell line RAW264.7 were incubated with a test compound at maximum final concentration of 2 μM for 24 hours. The macrophages were then activated by adding lipopolysaccharide (100 ng/mL) and γ-interferon (100 units/mL). The nitrogen monoxide (NO) production of activated macrophages was measured indirectly by quantifying nitrogen dioxide (NO₂) in the media according to Green L. et al., Anal. Biochem. 126, 131-138 (1982). Compounds of this invention were found to inhibit cholesterol synthesis of the cell to various extent.

Macrophage-Foam Cell Transformation

Constitutive expression of rat UR and human RXRa gene by retroviral systems in RAW264.7 transformed these cells into foam-cell-like morphology and integrated into clamps while increasing cell sizes and undergoing apoptosis. Foam cells originated from macrophages are the major components in pathological plaques formed on the inner wall of blood vessels which are a characteristic feature in atherosclerosis. Compounds of this invention were shown to be able to suppress the progression of macrophage-foam cell transformation at different stages, and thus can be used in the treatment or prevention of atherosclerosis.

Adipocyte Differentiation

Constitutive expression of rat UR gene in murine fibroblasts 3T3-L1 was done by using retroviral systems. Full-length rat UR cDNA was inserted into retroviral expression vector MV7. Infected 3T3-L1 cells that are G418-resistant were treated with 5 μg/ml insulin, 250 nM dexamethacine, and 0.5 mM 1-methyl-3-isobutylxanthine (MIX) to induce adipocyte differentiation. A control experiment was done by inserting human UR cDNA into MV7 in the antisense orientation. Cells infected with hUR-antisense constructs and parent 3T3-L1 cells were also treated with the same insulin cocktail under same cell density. Cells infected with rUR were shown to accumulate more Red oil O positive lipid drops than parent cells, while cells infected with hUR antisense were shown to have less Red oil O positive lipid drops.

Erythrocyte Differentiation

Constitutive expression of rat UR gene in murine NN10, IW32.1 or IW201 was done by using retroviral systems. Full-length rat UR cDNA was inserted into retroviral expression vector MV7. Infected cells that were G418-resistant were cultured up to 5 days to induce erythrocyte differentiation. A control experiment was done by using parent MV7 vector. NN10, IW32.1 or IW201 cells infected with parent MV7 construct were also treated with G418 in parallel under same cell density. More cells infected with rUR were shown to accumulate hemoglobin protein (stained with benzidine) than parent or control cells. When IW32.1/rUR cells were cultured on fibronectin-coated plates, some cells differentiated into mature enucleated reticulocytes.

Animal Studies

Male Sprague-Dawley rats that were 50 days old were fed a regular chow diet and tap water ad libitum for 1 week during acclimatization, and then randomly divided into groups that were given different dietary treatments. Both control and treatment groups were initially fed ad libitum a cholesterol-enriched diet, which was prepared by adding 2% cholesterol and 1% 3α,7α,12α-trihydroxy-5β-cholanoic acid to the regular chow diet. The treatment group received the same diet supplemented with 0.03% test steroid derivative. Rats were fasted overnight before determining body and liver weight and drawing blood from the tail vein for serum total cholesterol measurements. Total cholesterol was determined enzymatically with a diagnostic kit (Sigma, St. Louis, Mo.) on day 0 and 7. Average food consumption was 20-25 g/rat/day and average feces production was 9 g/rat/day. There was no statistical difference between control and treatment groups for food consumption and feces production. The dose for test steroid derivative in the treatment group was 40-50 mg/kg/day. Rats on high cholesterol/bile acid diet and treated with a trifluoromethyl conjugated 3α,6α-dihydroxy-5β-cholanoic acid had a 20% drop (p<0.05) in the serum total cholesterol compared with the level in untreated animals (Table 1). Food consumption, body and liver weight were similar in the control and treatment groups. In another experiement, rats were made hypercholesterolemic with a high cholesterol/cholic acid diet and then treatment with the same trifluoromethyl conjugated 3α,6α-dihydroxy-5β-cholanoic acid again lowered the serum total cholesterol by 20% compared with untreated animals.

Anti-Proliferative Effects on Prostate and Breast Cancer Cells

General

A monoclonal anti-p27 antibody is obtained from Transduction Laboratories (Lexington, Ky.). Polyclonal anti-Skp2 and anti-p21 goat IgGs are obtained from Santa Cruz Biotechnology (Santa Cruz, Calif.). A monoclonal anti-actin antibody is obtained from Chemicon (Temecula, Calif.). A monoclonal anti-c-Myc antibody 9E10 is prepared from hybridoma obtained from the American Type Culture Collection (ATCC) (Manassas, Va.). Human prostate cancer DU-145, PC-3, human breast cancer MCF-7 and MDA-MB435S cells are obtained from ATCC and maintained in Dulbecco's modified Eagle medium supplemented with 10% fetal bovine serum.

Data are presented as the mean±standard deviation or standard error of three experiments or are representative of experiments repeated at least three times.

Inhibition of Human Prostate Cancer Cell Growth

To determine whether the LXR agonists described herein inhibit human prostate cancer growth, androgen-dependent LNCaP 104-S cells and androgen-independent LNCaP 104-R1 cells were treated with 22(R)-hydroxycholesterol and 24(S)-hydroxycholesterol.

Androgen-dependent LNCaP 104-S cells and androgen-independent LNCaP 104-R1 cells were maintained and cultured as described in, e.g., Kokontis J, Takakura K, Hay N, and Liao S. “Increased androgen receptor activity and altered c-myc expression in prostate cancer cells after long-term androgen deprivation,” Cancer Res. 1994; 54: 1566-73; and Kokontis J M, Hay N, and Liao S. “Progression of LNCaP prostate tumor cells during androgen deprivation: hormone-independent growth, repression of proliferation by androgen, and role for p27Kip1 in androgen-induced cell cycle arrest,” Mol Endocrinol 1998; 12: 941-53.

The 104-S and 104-R1 cells were grown for 4 days in the presence of 22(R)-hydroxycholesterol and 24(S)-hydroxycholesterol at the concentrations listed in Table 1. Cell number was analyzed by measuring DNA content with the fluorescent dye Hoechst 33258 (SIGMA, St. Louis, Mo.) as described in, e.g., Rago R, Mitchen J, and Wilding G. “DNA fluorometric assay in 96-well tissue culture plates using Hoechst 33258 after cell lysis by freezing in distilled water,” Anal Biochem. 1990; 191: 31-4. The growth data is per cent of vehicle control (see Tables 1 and 2).

TABLE 1
Growth Data for 22(R)-hydroxycholesterol

| | 1 μM | 2.5 μM | 5 μM | 10 μM |
|---|---|---|---|---|
| 104-S Growth | 96% | 97% | 90% | 48% |
| 104-R1 Growth | 98% | 92% | 90% | 70% |

TABLE 2
Growth Data for 24(S)-hydroxycholesterol

| | 1 μM | 2.5 μM | 5 μM | 10 μM |
|---|---|---|---|---|
| 104-S Growth | 91% | 78% | 62% | 41% |
| 104-R1 Growth | — | 90% | 70% | 38% |

Expression of LXR Target Genes

The expression of LXR-target genes is analyzed by real-time quantitative PCR. Total RNA is isolated using the TRIZOL Reagent (Invitrogen, Carlsbad, Calif.) and is treated with DNase I (DNA-free, Ambion, Austin, Tex.). Reverse transcription is performed with random hexamers and Moloney murine leukemia virus reverse transcriptase (Omniscript, QIAGEN, Valencia, Calif.). The TaqMan primer/probe is designed using Primer Express (Applied Biosystems, Foster City, Calif.). The 5′-end of the probe is labeled with the reporter-fluorescent dye, FAM. The 3′-end of probe is labeled with the quencher dye, TAMRA. The sequences of primers and probes are as follows: ABCA1 primers, 5′-TGTCCAGTCCAGTAATGGTTCTGT-3′ and 5′-AAGCGAGATATGGTCCGGATT-3′, ABCA1 probe 5′-ACACCTGGAGAGAAGCTTTCAACGAGACTAACC-3′; SREBP-1c primers, 5′-GGTAGGGCCAACGGCCT-3′ and 5′-CTGTCTTGGTTGTTGATAAGCTGAA-3′, SREBP-1c probe, 5′-ATCGCGGAGCCATGGATTGCACT-3′; p27 primers, 5′-CCGGTGGACCACGAAGAGT-3′ and 5′-GCTCGCCTCTTCCATGTCTC-3′, p27 probe, 5′-AACCCGGGACTTGGAGAAGCACTGC-3′, respectively. Real-time PCR is performed on an ABI PRISM 7700 system (Applied Biosystems) using the QuantiTect Probe RT-PCR protocol (QIAGEN). The Ribosomal RNA Control Kit (Applied Biosystems) is used to normalize transcript levels between samples.

Effect of LXR Agonists on Cell Cycle Distribution

The effect of LXR receptor agonists on cell cycle distribution in the LNCaP sublines 104-S and 104-R1 is examined using flow cytometry of propidium iodide-stained cells. Cells are seeded at 5×10⁵ cells in 6 cm dishes. Cells are collected and fixed in 70% ethanol/30% phosphate buffered saline (PBS) overnight at −20° C. Fixed cells are washed with PBS, treated with 0.1 mg/ml RNase A in PBS for 30 minutes and then suspended in 50 μg/ml propidium iodide in PBS. Cell cycle profiles and distributions are determined using a BD Facscan flow cytometer (BD Biosciences, San Jose, Calif.). Cell cycle distribution is analyzed using ModFit LT software (Verity Software House, Topsham, Me.).

Since the expression level of the cell cycle dependent kinase inhibitor p27 is increased when LNCaP cells are arrested and in G1 phase (see, e.g., Kokontis J M, Hay N, and Liao S. “Progression of LNCAP prostate tumor cells during androgen deprivation: hormone-independent growth, repression of proliferation by androgen, and role for p27Kip1 in androgen-induced cell cycle arrest,” Mol. Endocrinol. 1998; 12: 941-53), Western blotting can be performed to examine the effect of LXR receptor agonists on p27 expression. Protein extracts are prepared by lysing PBS-washed cells on the dish with Laemmli gel loading buffer without bromophenol blue dye. Protein concentration is determined with the Bradford reagent (Bio-Rad Laboratories, Hercules, Calif.) using a bovine serum albumin standard. Proteins are separated on 6% polyacrylamide gels containing SDS. Electrophoresis and blotting are performed as described in, e.g., Kokontis J M, Hay N, and Liao S. “Progression of LNCaP prostate tumor cells during androgen deprivation: hormone-independent growth, repression of proliferation by androgen, and role for p27Kip1 in androgen-induced cell cycle arrest,” Mol. Endocrinol. 1998; 12: 941-53. Measurement of actin expression is used as a loading control.

Other molecules believed to be involved in LNCaP cell proliferation can also be analyzed by Western analysis (see e.g., Kokontis J, Takakura K, Hay N, and Liao S. “Increased androgen receptor activity and altered c-myc expression in prostate cancer cells after long-term androgen deprivation,” Cancer Res. 1994; 54: 1566-73; and Kokontis J M, Hay N, and Liao S. “Progression of LNCaP prostate tumor cells during androgen deprivation: hormone-independent growth, repression of proliferation by androgen, and role for p27Kip1 in androgen-induced cell cycle arrest,” Mol. Endocrinol. 1998; 12: 941-53).

To demonstrate that the level of p27 is functionally involved in LXR receptor agonist-induced cell cycle arrest, p27-knockdown 104-R1 cells are generated using an expression plasmid generating RNAi for p27. The RNAi sequence is designed by using the AA scanning program from OligoEngine (Seattle, Wash.). DNA coding for an RNAi for human p27 is prepared using the following oligonucleotides: 5′-GATCCCCGCACTGCAGAGACATGGAATTCAAGAGATTCCATGTCTCTGCAGT GCTTTTTGGAAA-3′ and 5′-AGCTTTTCCAAAAAGCACTGCAGAGACATGGAATCTCTTGAATTCCATGTCTC TGCAGTGCGGG-3′. These 64-mer oligonucleotides are annealed and ligated into the pH1RP vector (see, e.g., Fukuchi J, Hiipakka R A, Kokontis J M, Nishimura K, Igarashi K, and Liao S. “TATA-binding protein-associated factor 7 regulates polyamine transport activity and polyamine analog-induced apoptosis,” J. Biol. Chem. 2004; 279: 29921-9). The p27-RNAi expression plasmid is stably transfected into 104-S cells using Effectene (QIAGEN) and selection for G418 resistance.

Inhibition of Breast and Other Prostate Cancer Cell Growth

The effect of LXR receptor agonists on the growth of various breast and other prostate cancer cell lines can also be determined. These cell lines can include: human prostate cancer PC-3 cells, breast cancer MCF-7 and MDA-MB435S cells, and human prostate cancer LNCaP and DU-145 cells.

Using retroviral infection, human LXRα in MDA-MB435S cells are ectopically expressed. Ectopic expression of LXRα is achieved by infecting MDA-MB534S cells with pLNCX2 retrovirus (Clonetech, Palo Alto, Calif.) carrying the human LXRα cDNA (see, e.g., Janowski B A, Willy P J, Devi T R, Falck J R, and Mangelsdorf D J. “An oxysterol signalling pathway mediated by the nuclear receptor LXR alpha,” Nature 1996; 383: 728-31). Retrovirus is generated using the Phoenix-ampho packaging cell line (G. Nolan, Stanford University).

Athymic Nude Mice Study

To determine whether LXR receptor agonists have anti-proliferation effects in vivo, a candidate LXR receptor agonist is tested against LNCaP 104-S xenografts in athymic nude mice. Six to eight week old male BALB/c nu/nu mice (NCI-Frederick, Frederick, Md.) are injected subcutaneously (see, e.g., Umekita Y, Hiipakka R A, Kokontis J M, and Liao S. “Human prostate tumor growth in athymic mice: inhibition by androgens and stimulation by finasteride,” Proc. Natl. Acad. Sci. U. S. A. 1996; 93: 11802-7) with 10⁶ LNCaP 104-S cells suspended in 0.25 ml of Matrigel (BD Bioscience, Bedford, Mass.). Tumors are measured weekly using a caliper and their volumes are calculated using the formula length×width×height×0.52. In some embodiments, the initial tumor volumes can be about 90 mm³ prior to treatment. The candidate LXR receptor agonist is administered via daily oral gavage using sesame oil vehicle at a dose of about 10 mg/kg body weight per day.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Accordingly, other embodiments are within claims.

What is claimed is:

1. A method for treating prostate cancer, the method comprising administering to a subject in need thereof an effective amount of a Liver X receptor agonist having formula (II): 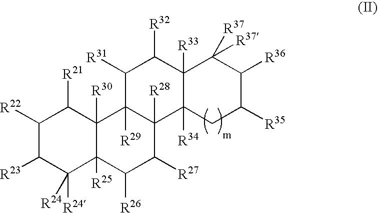
wherein:
each of R²¹, R²², R^{24′}, R³¹, and R^{37′}, independently, is hydrogen, halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, —C(O)NR^{a}R^{b}; or —NR^{d}C(O)R^{c};
each of R²³, R²⁴, R²⁶, R²⁷, R³², R³⁵, R³⁶, independently, is hydrogen, halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, —C(O)NR^{a}R^{b}; or —NR^{d}C(O)R^{c}; or R23 together with R²⁴ is a bond, R²⁴ together with R²⁵ is a bond, R²⁵ together with R²⁶ is a bond, R²⁷ together with R²⁸ is a bond, R³² together with R³³ is a bond, or R³⁵ together with R³⁶ is a bond;
each of R²⁵, R²⁸, and R³³, independently, is hydrogen or C₁-C₁₂ alkyl; or R²⁵ together with R²⁴ is a bond, R²⁵ together with R²⁶ is a bond, R²⁸ together with R²⁷ is a bond, or R³³ together with R³² is a bond;
each of R²⁹, R³⁰, and R³⁴, independently, is hydrogen or C₁-C₁₂ alkyl;
R³⁷ is C₁-C₂₀ alkyl substituted with hydroxy, oxo, NR^{a}R^{b}, C₁-C₁₂ alkoxy, —C(O)R^{c}, —OC(O)R^{c}, or —NR^{d}C(O)R^{c}; or —C(O)NR^{a}R^{b};
each of R^{a}, R^{b}, and R^{d}, at each occurrence is, independently, hydrogen or C₁-C₁₀ alkyl;
R^{c}, at each occurrence is, independently, C₁-C₁₂ alkyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₆-C₁₆ aryl; or heteroaryl including 5-16 atoms; and
m is 0, 1,or2;
provided that at least one of R²³ and R24, R24 and R²⁵, R²⁶ and R26, R27 and R²⁸, R³² and R³³, or R³⁵ and R³⁶, together is a bond; or a salt thereof.

2. The method of claim 1, wherein the prostate cancer is an androgen-dependent prostate cancer.

3. The method of claim 1, wherein the prostate cancer is resistant to androgen deprivation and/or antiandrogen therapy.

4. The method of claim 3, wherein the prostate cancer is an androgen-independent prostate cancer.

5. The method of claim 4, wherein the androgen-independent prostate cancer is a hormone-refractory prostate cancer.

6. The method of claim 1, wherein the subject has at least one prostate cancer tumor that is resistant to androgen deprivation and/or antiandrogen therapy.

7. The method of claim 6, wherein the subject has at least one androgen-independent prostate cancer tumor.

8. The method of claim 6, wherein the subject is substantially free of androgen-dependent prostate cancer tumors.

9. The method of claim 1, wherein the Liver X receptor agonist is orally administered.

10. The method of claim 1, wherein the Liver X receptor is LXRα or LXRβ.

11. The method of claim 1, wherein R²⁵ together with R²⁶ is a bond.

12. The method of claim 1, wherein m is 0.

13. The method of claim 1, wherein R²³ is halo, hydroxy, oxo, —O-sulfonic acid, C₁-C₁₂ alkyl, C₂-C₁₂ alkenyl, C₂-C₁₂ alkynyl, NR^{a}R^{b}, C₁-C₁₂ alkoxy, C₁-C₁₂ haloalkoxy, C₆-C₁₆ aryloxy, —C(O)R^{c}, —C(O)OR^{c}, —OC(O)R^{c}, —C(O)NR^{a}R^{b}; or —NR^{d}C(O)R^{c}.

14. The method of claim 13, wherein R²³ is hydroxy, oxo, —O-sulfonic acid, NR^{a}R^{b}, C₁-C₁₂ alkoxy, C₁-C₁₂ haloalkoxy, C₆-C₁₆ aryloxy, —OC(O)R^{c}, or —NRC(O)R^{c}.

15. The method of claim 14, wherein R²³ is hydroxy, oxo, —O-sulfonic acid, C₁-C₁₂ alkoxy, C₁-C₁₂ haloalkoxy, C₆-C₁₆ aryloxy, or —OC(O)R^{c}.

16. The method of claim 15, wherein R²³ is hydroxy.

17. The method of claim 13, wherein R²³ is in the β-configuration.

18. The method of claim 11, wherein each of R²¹, R²², R²⁴, R^{24′}, R²⁷, R³¹, R³², R³⁵, R³⁶ , and R^{37′}, independently, is hydrogen, hydroxy, or oxo, and each of R²⁸, R²⁹, R³⁰, R³³, and R³⁴ is hydrogen or C₁-C₆ alkyl.

19. The method of claim 18, wherein each of R²¹, R²², R²⁴, R^{24′}, R²⁷, R²⁸, R29, R³¹, R³², R³⁴, R³⁵, R³⁶, and R^{37′} is hydrogen and each of R³⁰ and R³³ is C₁-C₆ alkyl.

20. The method of claim 18, wherein R²³ is hydroxyl, each of R²¹, R²², R²⁴, R^{24′}, R²⁷, R²⁸, R²⁹, R³¹, R³², R³⁴, R³⁵, R³⁶, R^{37′} is hydrogen, and each of R³⁰ and R³³is C₁-C₆ alkyl.

21. The method of claim 20, wherein each of R³⁰ and R³³ is CH₃.

22. The method of claim 20, wherein R²³ is in the β-configuration.

23. The method of claim 1, wherein R³⁷ is C₁-C₂₀ alkyl substituted with hydroxy.

24. The method of claim 23, wherein R³⁷ is C₆-C₂₀ alkyl substituted with hydroxy.

25. The method of claim 24, wherein R³⁷ is C₈-C₁₆ alkyl substituted with hydroxy.

26. The method of claim 25, wherein R³⁷ is —CH(CH₃)CH(OH)CH₂CH₂CH(CH₃)₂.

27. The method of claim 25, wherein R³⁷ is —CH(CH₃)CH₂CH₂CH(OH)CH(CH₃)₂.

28. The method of claim 1, wherein the Liver X receptor agonist is 22(R)-hydroxycholesterol.

29. The method of claim 1, wherein the Liver X receptor agonist is 24(S)-hydroxycholesterol.

30. The method of claim 1, wherein the compound of formula (I) is administered with a pharmaceutically acceptable carrier or adjuvant.

31. The method of claim 1, wherein the salt is a pharmaceutically acceptable salt.


SEQUENCE LISTING (22 sequences)

SEQ ID NO: 1 - DNA, 16 bp, Artificial Sequence
  Synthetically generated oligonucleotide
aggtcaagccaggtca

SEQ ID NO: 2 - DNA, 57 bp, Artificial Sequence
  Synthetically generated oligonucleotide
gtatcgccggaattcnnnttgnnnnnnttgttgnnnnnntaagtcgactctagagcc

SEQ ID NO: 3 - DNA, 57 bp, Artificial Sequence
  Synthetically generated oligonucleotide
ggctctagagtcgacttannnnnncaacaannnnnncaannngaattccggcgatac

SEQ ID NO: 4 - DNA, 57 bp, Artificial Sequence
  Synthetically generated oligonucleotide
gtatcgccggaattcatcttgcacagattgttgcaagaataagtcgactctagagcc

SEQ ID NO: 5 - DNA, 57 bp, Artificial Sequence
  Synthetically generated oligonucleotide
ggctctagagtcgacttattcttgcaacaatctgtgcaagatgaattccggcgatac

SEQ ID NO: 6 - DNA, 60 bp, Artificial Sequence
  Synthetically generated oligonucleotide
gtatcgccggaattcnnnttgnnnnnntggttgttgnnnnnntaagtcgactctagagcc

SEQ ID NO: 7 - DNA, 60 bp, Artificial Sequence
  Synthetically generated oligonucleotide
ggctctagagtcgacttannnnnncaacaaccannnnnncaannngaattccggcgatac

SEQ ID NO: 8 - protein, 5 aa, Artificial Sequence
  Synthetically generated peptide
LXXLL

SEQ ID NO: 9 - protein, 6 aa, Artificial Sequence
  Synthetically generated peptide
LXXWLL

SEQ ID NO: 10 - DNA, 26 bp, Artificial Sequence
  Synthetically generated oligonucleotide
ttcaggtcacaggaggtcagagagct

SEQ ID NO: 11 - DNA, 27 bp, Artificial Sequence
  Synthetically generated oligonucleotide
tcgagtctggtacagggtgttcttttg

SEQ ID NO: 12 - DNA, 24 bp, Artificial Sequence
  Primer
tgtccagtccagtaatggttctgt

SEQ ID NO: 13 - DNA, 21 bp, Artificial Sequence
  Primer
aagcgagatatggtccggatt

SEQ ID NO: 14 - DNA, 33 bp, Artificial Sequence
  Probe sequence
acacctggagagaagctttcaacgagactaacc

SEQ ID NO: 15 - DNA, 17 bp, Artificial Sequence
  Primer
ggtagggccaacggcct

SEQ ID NO: 16 - DNA, 25 bp, Artificial Sequence
  Primer
ctgtcttggttgttgataagctgaa

SEQ ID NO: 17 - DNA, 23 bp, Artificial Sequence
  Probe sequence
atcgcggagccatggattgcact

SEQ ID NO: 18 - DNA, 19 bp, Artificial Sequence
  Primer
ccggtggaccacgaagagt

SEQ ID NO: 19 - DNA, 20 bp, Artificial Sequence
  Primer
gctcgcctcttccatgtctc

SEQ ID NO: 20 - DNA, 25 bp, Artificial Sequence
  Probe sequence
aacccgggacttggagaagcactgc

SEQ ID NO: 21 - DNA, 64 bp, Artificial Sequence
  Synthetically generated oligonucleotide
gatccccgcactgcagagacatggaattcaagagattccatgtctctgcagtgctttttggaaa

SEQ ID NO: 22 - DNA, 64 bp, Artificial Sequence
  Synthetically generated oligonucleotide
agcttttccaaaaagcactgcagagacatggaatctcttgaattccatgtctctgcagtgcggg